(19) United States
(12) Patent Application Publication
(10) Pub. No.: US 2023/0218316 A1
(43) Pub. Date: Jul. 13, 2023

(54) PERCUTANEOUS INVASIVE INSTRUMENT GUIDE

(71) Applicant: The Feinstein Institutes for Medical Research, Manhasset, NY (US)

(72) Inventors: Ernesto Pompeo MOLMENTI, Kings Point, NY (US); Lucy Dolores HALPIN, Dublin (IE); Derek YOUNG, Wexford (IE); Kate GILLEN, Dublin (IE); Laura CADEN, Dublin (IE)

(21) Appl. No.: 17/925,572

(22) PCT Filed: Feb. 28, 2022
(86) PCT No.: PCT/US2022/018158

§ 371 Date: Nov. 15, 2022

Related U.S. Application Data
(60) Provisional application No. 63/154,977, filed on Mar. 1, 2021.

(51) Int. Cl. A61B 17/34; A61B 34/30

(52) U.S. Cl. CPC A61B 17/3403; A61B 34/30; A61B 90/11

(57) ABSTRACT

An introducer guide includes a guiding assembly to guide insertion an instrument, such as a biopsy needle, through a selected insertion point on a patient's body and along a selected insertion path. An imaging system, such as a CT scanner, is used to visualize portions of the guiding assembly in relation to the patient's tissues as the insertion path is adjusted. The guiding assembly includes a semicircular arch connected with a base plate by sliding hinges with a center of curvature of the arch centered on the insertion point. A guide body is slidably connected with the arch. Rotation of the arch about the hinges adjusts a first angle of the insertion path. Motion of the guide body along the arch adjusts a second angle of the insertion path. Linkages, such as linear actuation cables, rotary cables, or pneumatic or hydraulic actuators, connect the arch and guide body with remote operators. A practitioner aligns the guide assembly with the insertion point and fixes the base to the patient's skin. The practitioner uses the remote operators to adjust the orientation of the insertion path while visualizing the insertion path using the imaging system. The length of the cables is selected to allow the practitioner to adjust the guiding assembly at a safe distance from ionizing radiation emitted by the imaging system.

### BACKGROUND

### Field

This disclosure relates to a device for guiding the insertion of a needle, introducer, or other medical instrument, into a patient during a medical procedure. More particularly, the disclosure relates to a device that provides a guide that can be accurately adjusted to define an insertion path of a medical device such as a biopsy needle, that can be locked into position so that the insertion path remains stable during the medical procedure, and that can be adjusted remotely from the site of insertion enabling a practitioner to operate the device outside of the confines of an imaging system such as a CT scanner, fluoroscope, MM scanner and the like.

Some medical procedures require a needle or other medical instrument to be inserted into a patient and accurately guided to a particular location in the body. For example, diagnostic biopsy procedures are often performed by inserting a needle into a mass within the patient's body to retrieve a sample of tissue to determine a pathology. Therapeutic procedures may also be performed using an instrument inserted along a particular trajectory to apply medications, surgical operations, or to deliver destructive energy, such as thermal ablation, to tissue at a specific site. Examples of procedures that may be performed using embodiments of the disclosure include, but are not limited to, kyphoplasty/vertebroplasty, bone biopsies, brachytherapy, radio-frequency ablation, denervation, spine injections, percutaneous cryotherapy, ascitic tap biliary drainage, pleural aspiration, orthopedic procedures including placement of k-wires and the like, bone marrow biopsies, bone marrow transfusions, and percutaneous nephrolithotomy.

To accurately locate the needle or medical instrument, imaging techniques are often used before and during the procedure to guide the instrument to the area of tissue to be examined or treated. Imaging may be done using ionizing x-ray radiation, for example, by a CT scanner, Cone Beam CT scanner, or fluoroscope. Using these imaging devices subjects the patient and medical personnel to ionizing radiation, which can be hazardous, especially for physicians, nurses and other professionals that perform procedures repeatedly and may be exposed to ionizing radiation each time a procedure is performed. Thus, there is a need for a device that enables procedures requiring guided insertion of a needle or other medical instrument that minimizes exposure of the patient and medical personnel to the radiation used for imaging.

Imaging systems such as CT scanners and Mill scanners often provide a very confined space around the patient in the area where an image is being captured. This limited space may present difficulties for practitioners where a needle or other instrument needs to be directed to a portion of tissue identified using the imaging device. There may be little space between the patient's body and the bore of the imaging machine for the practitioner's hands and medical instruments. The lack of space to work within the imager may be exacerbated where the patient has a large frame or is obese. Thus, there is a need for a device that enables needles and other medical instruments to be guided using imagers that minimizes the space required within the imager. There is also a need for such a device that can be adjusted to define an insertion path while the patient is in an imaging system and that stably maintains that insertion path once the patient is removed from the imaging device. This allows procedures to be performed without having the patient confined inside the imaging system.

In addition, ferromagnetic materials generally cannot be used near MRI scanners. Such materials may distort the magnetic field, reducing the quality of the imaging. In some cases, metallic objects present a hazard to the patient and to medical personnel due to the high magnetic field strength generated by MM scanners. Thus, there is a need for a device that enables needles and other medical instruments to be guided using magnetic resonance imaging (MM) that does not include ferromagnetic components.

### SUMMARY

The present disclosure relates to a device for guiding the insertion of needles and other medical instruments that addresses these and other difficulties.

According to one aspect of the disclosure, there is provided a medical instrument guide that is used by a medical practitioner to establish an insertion path and that can be adjusted at a distance from the area subject to ionizing radiation generated by an imaging device.

According to another aspect, there is provided a medical instrument guide that is formed from non-ferromagnetic materials that does not distort magnetic fields used by imaging equipment.

According to another aspect, there is provided a medical instrument guide made from radio-transparent or radio-translucent materials to allow an imaging system to generate an unobstructed view of a patient's tissues while the guide is being adjusted to select an insertion path.

According to a further aspect, there is provided a medical instrument guide that stably maintains the selected insertion path once the patient is removed from the imaging device.

According to another aspect, there is provided a medical instrument guide that includes radio-opaque features to illustrate the location of the guide relative to a desired insertion point and to illustrate the insertion path of the guide and the relation of that path with the patient's tissue when the guide is visualized using a medical imaging device.

According to a further aspect, there is provided a medical instrument guide that can be positioned at precise angular orientations to adjust the path of inserting of a needle or other medical instrument.

According to a further aspect, the medical instrument guide holds the angular orientation of the insertion path in a stable manner. This allows the insertion path to be set at a fixed orientation while a patient is positioned within an imaging device and for a medical procedure to be performed after the patient is moved away from the imaging system. This also allows the insertion path to be set at a fixed orientation by one practitioner, for example, a nurse or radiologist, and for the medical procedure to be performed by another practitioner, for example, a surgeon.

According to a still further aspect, there is provided a medical instrument guide that defines an insertion point co-planar with the patient's skin surface and that maintains the same insertion point regardless of the angle of the path of insertion relative to the patient's tissue.

According to one embodiment there is provided medical device introducer guide that includes a guide assembly comprising a base adapted to be affixed to an organism relative to an insertion point and an arch connected with the support. The arch has a semicircular curvature, the curvature having a radius of curvature centered on the insertion point. The insertion point is co-planar with an outer surface of the organism. A guide body is slidably disposed on the arch. The guide body includes a bore. An axis of the bore defines an insertion path. The insertion path has an orientation and intersects the insertion point. The introducer guide includes a remote operator and a linkage connected with the remote operator and the guide assembly. Motion of the remote operator is communicated by the linkage to one or more of the arch and the guide body to vary the orientation of the insertion path.

One or more hinges may connect the arch with the base. The hinges allow the arch to rotate about axis of rotation parallel with the base while the axis of rotation intersects the insertion point. The hinge may comprise two sliding hinges. The sliding hinges may each comprise a semicircular support surface fixed to the base and having a hinge radius of curvature, where the hinge radius of curvature is centered on the axis of rotation. The sliding hinges may also comprise a slider in sliding contact with the support surface, wherein the arch is fixed with the slider and extends from the slider in a direction radially away from the support surface. Rotation of the arch about the axis of rotation slides the slider along the support surface. Curvature of the slider may conform with the curvature of the support surface. The introducer guide may further comprise a retainer fixed with the base where the retainer has a semicircular inner surface that is concentric with the support surface, where an upper surface of the slider is in sliding contact with the retainer, and where the retainer holds the slider against the support surface.

The linkage may comprise a first cable. The first cable has a first shaft and a first sheath surrounding the first shaft. A distal end of the first sheath is fixed to the arch and a distal end of the first shaft is fixed to the guide body. The motion is communicated by movement of the first shaft relative to the first sheath to move the guide body along the arch to vary the orientation of the insertion path through a first angle.

The linkage may comprise a second cable. The second cable has a second shaft and a second sheath surrounding the second shaft. A distal end of the second sheath is fixed to the base and a distal end of the second shaft is fixed to the arch. The motion is communicated by movement of the second shaft relative to the second sheath to move the arch relative to the base and to vary the orientation of the insertion path through a second angle.

The remote operator may comprise a guide body operator having a first housing and a first sliding actuator. The first sliding actuator is adapted to slide in a distal and a proximal direction. The first sheath of the first cable is fixed with the first housing and the first shaft is fixed with the first sliding actuator. Motion of the first sliding actuator in the distal and proximal directions moves the guide body along the arch through the first angle. A second remote operator connected with the second shaft and second sheath of the second cable may be provided to move the arch relative to the base through the second angle.

The guide assembly may comprise a material with a first radio-opacity and the base may comprise one or more center alignment indicators shaped to indicate a direction relative to the insertion point. The center alignment indicators have a radio-opacity greater than the first radio-opacity. When viewed under x-ray radiation, the center alignment indicators show the position of the insertion point.

The guide body may comprise a plurality of path alignment indicators arranged co-linearly with the bore. The path alignment indicators have a radio-opacity different from the first radio-opacity. When viewed under x-ray radiation, the path alignment indicators show the orientation of the insertion path.

The base may comprise a lower plate and an upper plate. A bottom surface of the lower plate is adapted to be fix to the organism. An upper surface of the lower plate may comprise a rack gear disposed along at least part of a circular path centered on the insertion point. The upper plate is rotatably connected with the lower plate and the arch is fix to the upper plate and extends upward in a plane normal to the upper plate. A pinion gear is rotatably mounted to the upper plate. The pinion gear engages the rack gear. Rotation of the pinion gear causes the upper plate and the arch to rotate relative to the lower plate. The linkage may comprise a rotary cable. A distal end of the rotary cable is connected with the pinion gear. The remote operator may comprise a knob connected with a proximal end of the rotary cable. Rotation of the knob causes the upper plate and arch to rotate relative to the lower plate.

The linkages comprise one or more of a Bowden cable, a rotary control cable, a hydraulic cylinder, and a pneumatic cylinder.

The introducer guide may comprise one or more rotational position indicators, the rotational position indicators formed from a material with a radio-opacity greater than the first radio-opacity.

The linkage may comprise one or more universal joints.

The linkage may comprise a fluid-driven actuator. When fluid is moved into or out from the actuator, the actuator exerts force on the guide body to move the guide body to the selected location. The actuator may comprise a bellows or a piston slidably disposed in an internal cavity of the arch and the linkage may comprise a hose in fluid communication with the bellows or cavity and a fluid pump in fluid communication with the hose. Actuation of the pump moves fluid into or out from the actuator to move the guide body. The fluid may be a gas, a mixture of gasses, or a liquid. The actuator may also comprise a bellows and, in the absence of an internal pressure, the bellows assumes a first configuration to move the guide body. The pump may comprise a syringe or a squeeze bulb. Alternatively, the actuator is an electrically driven motor. The motor applies force to the arch and/or guide body to adjust the insertion path. The motor may be controlled remotely, for example, using a radiofrequency communication device.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a perspective view of a medical instrument guide according to an embodiment of the disclosure with guide assembly disposed on a human being imaged in an imaging device and being operated by a medical practitioner;

FIG. 2 is a perspective view of the medical instrument guide of FIG. 1;

FIG. 3A is a perspective view of the guide assembly of a medical instrument guide according to an embodiment of the disclosure;

FIG. 3B is an exploded view of the guide assembly of FIG. 3A;

FIG. 3C is a partial cross section view of the guide assembly of FIG. 3A;

FIG. 3D is another partial cross section view of the guide assembly of FIG. 3A;

FIG. 4A is a top view of a guide assembly according to an embodiment of the disclosure illustrating an adhesive patch to adhere the guide assembly to a patient;

FIG. 4B is a side view of the guide assembly of FIG. 4A;

FIGS. 4C and 4D show steps for adhering the guide assembly of FIG. 4A to a patient;

FIGS. 5A, 5B, and 5C are top views of guide assemblies including adhesive patches according to embodiments of the disclosure;

FIG. 6 is a top view of an adhesive patch according to a further embodiment of the disclosure;

FIG. 7A is a perspective view of a guide body according to embodiments of the disclosure;

FIG. 7B is a side view of the guide body of FIG. 7A;

FIG. 7C is a cross section view of the guide body of FIG. 7A;

FIG. 7D is a perspective view of a guide body according to other embodiments of the disclosure;

FIG. 8A is a cross section view of an insert for a guide body according to an embodiment of the disclosure;

FIGS. 8B and 8C are top views showing alternative embodiments for the insert of FIG. 8A;

FIG. 9A is a perspective view of an insert for a guide assembly according to a further embodiment of the disclosure;

FIG. 9B shows a detailed perspective view of membranes comprising the insert of FIG. 9A;

FIG. 9C is a top view of a membrane comprising the insert of FIG. 9A according to an alternative embodiment of the disclosure;

FIG. 9D is a cross section view of an insert for a guide body including membranes according to an alternative embodiment of the disclosure;

FIG. 10A is a perspective view of a guide assembly including an arch and guide body according to an embodiment of the disclosure;

FIG. 10B is another perspective view of the arch and guide body of FIG. 10A;

FIG. 10C is a cross section view of the arch and guide body of FIG. 10A;

FIG. 11 is a perspective view of an arch and guide body including position markings according to an embodiment of the disclosure;

FIGS. 12A and 12B are cross section views of an arch, a guide body, and a linkage between an actuator shaft and the guide body according to an embodiment of the disclosure;

FIG. 13A is a perspective view of a remote operator according to embodiments of the disclosure;

FIG. 13B is an exploded view of the remote operator of FIG. 13A;

FIGS. 14A and 14B are a top view and a side view, respectively, of a remote operator according to an embodiment of the disclosure;

FIG. 14C is an exploded view of the remote operator of FIGS. 14A and 14B;

FIG. 15 is a perspective view of a medical instrument guide according to an embodiment of the disclosure with guide assembly disposed on a human in an imaging device and with the guide being operated by a motorized operator;

FIG. 16A is a perspective view of a guide assembly including alignment features according to a further embodiment of the disclosure;

FIG. 16B is a top view of the base of the guide assembly of FIG. 16A;

FIGS. 17A, 17B, 17C, 17D, and 17E showing guide bodies including optical alignment features according to alternative embodiments of the disclosure;

FIG. 18A is a perspective view of the guide assembly of a medical instrument guide according to an embodiment of the disclosure;

FIG. 18B is a perspective view of the guide assembly of FIG. 18A with portions made transparent to illustrate an internal mechanism according to embodiments of the disclosure;

FIG. 18C is a side view of the guide assembly of FIG. 18A;

FIG. 18D is a schematic view of the guide assembly of FIG. 18A;

FIG. 19 is a perspective view of a remote operator according to embodiments of the disclosure;

FIGS. 20A and 20B show a guide assembly according to embodiments of the disclosure being oriented using a laser alignment system;

FIGS. 21A and 21B are fluoroscope images superimposed with a guide assembly according to embodiments of the disclosure showing the assembly being adjusted to a selected insertion path;

FIGS. 21C and 21D are fluoroscope images superimposed with a guide assembly according to an alternative embodiment of the disclosure showing the assembly being adjusted to a selected insertion path;

FIGS. 22A and 22B are perspective views of a guide assembly according to a further embodiment of the disclosure;

FIG. 23 is a perspective view of the guide assembly of FIGS. 22A and 22B coupled with a control arm according to an embodiment of the disclosure;

FIG. 24 is cross section view of the guide assembly of FIGS. 22A and 22B;

FIG. 25 is a perspective view of a guide assembly according to another embodiment of the disclosure;

FIG. 26 is a perspective view of a medical instrument guide according to another embodiment of the disclosure;

FIG. 27 is a cross section view of the guide assembly of the medical instrument guide of FIG. 26;

FIGS. 28 and 29 are a perspective view and a cross section view, respectively, of a bellows used with the guide assembly of FIG. 26;

FIGS. 30 and 31 are a perspective view and a cross-sectional view of a medical instrument guide according to another embodiment of the disclosure;

FIG. 32 is a perspective view of a medical instrument guide according to another embodiment of the disclosure;

FIGS. 33A and 33B are partial cross section views of the guide assembly of the medical instrument guide of FIG. 32;

FIG. 34 is a perspective view of a syringe actuator according to an embodiment of the disclosure;

FIG. 35 is a perspective view of a guide assembly according to another embodiment of the disclosure;

FIG. 36 is a cross section view of the guide assembly of FIG. 35;

FIG. 37 is a partial cross section side view of the guide assembly of FIG. 35; and

FIGS. 38, 39, and 40 are perspective and elevation views of a guide assembly according to another embodiment of the disclosure.

### DETAILED DESCRIPTION

For purposes of this disclosure, the terms “distal,” “distally,” “distal of” and the like will be used throughout this disclosure to refer to the direction or relative position away from the operator of the device and toward the body of a patient being treated using the device. The terms “proximal,” “proximally,” “proximal of” and the like will be used throughout this disclosure to refer to the direction toward the operator of the device and away from the body of a patient being treated using the device.

Embodiments are described in terms of treatment of a human patient. The disclosure is not limited to devices to treat humans and is applicable to perform veterinary procedures on animals. Embodiments of the disclosure are not limited to providing medical treatment and are applicable to performing procedures on cadavers, for example, during an autopsy, or for orienting an insertion path of an instrument relative to an inanimate object.

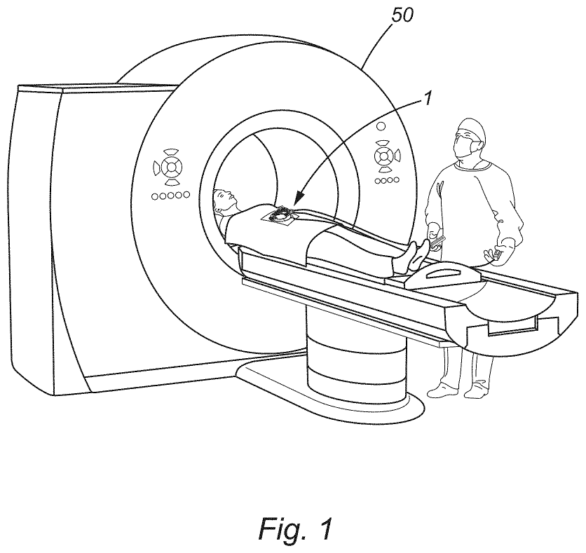

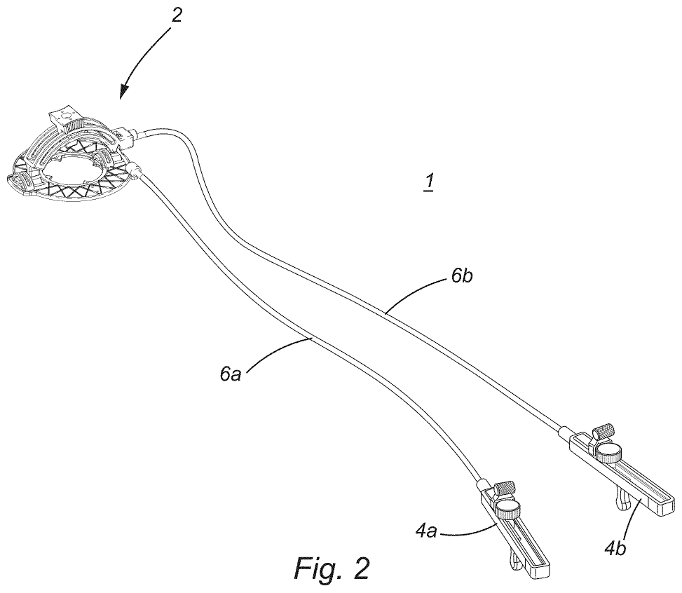

FIGS. 1 and 2 show an invasive medical instrument guide 1 according to one embodiment of the disclosure. Guide 1 includes guide assembly 2 that is adapted to be positioned on a human patient within an imaging device 50. Remote operators 4a and 4b are used by a practitioner to operate the guide assembly, as will be explained below. The imaging device may be a CT scanner, Cone Beam CT scanner, fluoroscope, MRI scanner, and the like. The imaging device is used to determine an insertion path of a medical instrument such as an introducer, a biopsy needle, a laparoscopic instrument, and the like.

For purposes of illustration, embodiments will be described with regard to apparatus and methods to guide insertion of a needle. The disclosure is not limited to guiding needles. The disclosure is applicable to insertion of any medical instrument that needs to be guided along a preselected insertion path into the body. Likewise, the present disclosure encompasses devices and methods for guiding other therapeutic modalities along a preselected path into a patient's tissue, for example, directing laser light, directing a collimated beam of ionizing radiation, and the like.

As shown in FIGS. 1 and 2, remote operators 4a and 4b are located outside the imaging device 50, or outside of the imaging field of the fluoroscope or other imaging equipment. The remote operators are connected with the guide assembly 2 by a linkage such as by cables 6a and 6b. As will be explained below, a medical practitioner operates the remote operators 4a and 4b via the linkages to adjust the guide assembly 2 with the aid of the imager 50 to select the insertion path of an instrument. Providing control of the medical instrument guide remote for the imager 50 reduces the practitioner's exposure to harmful radiation. Remote operation also reduces the amount of space required within the imager 50 because clearance does not need to be provided for the practitioner's hands. Also, by making the adjustment of the insertion path more convenient for the practitioner, the time the patient needs to be exposed to ionizing radiation, for example, under a fluoroscope, may be reduced, thus reducing the patient's exposure to ionizing radiation.

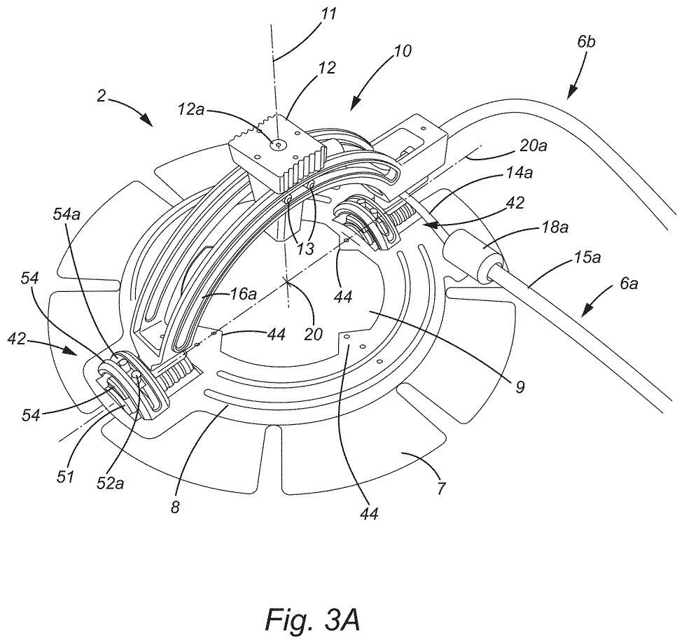

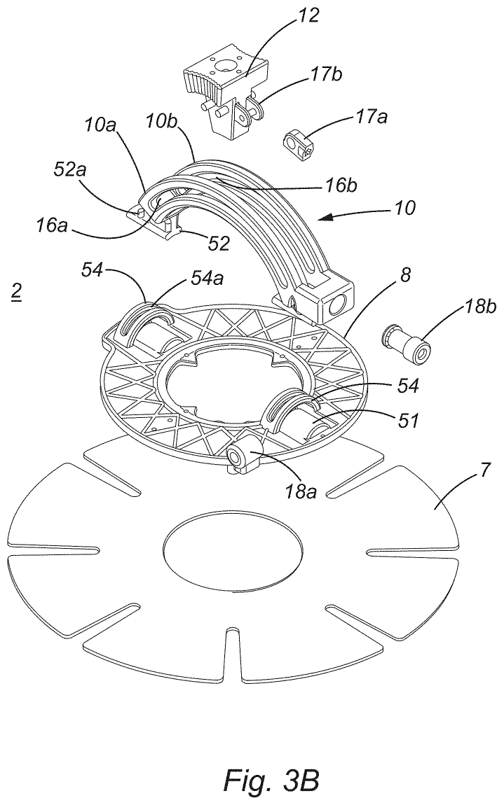

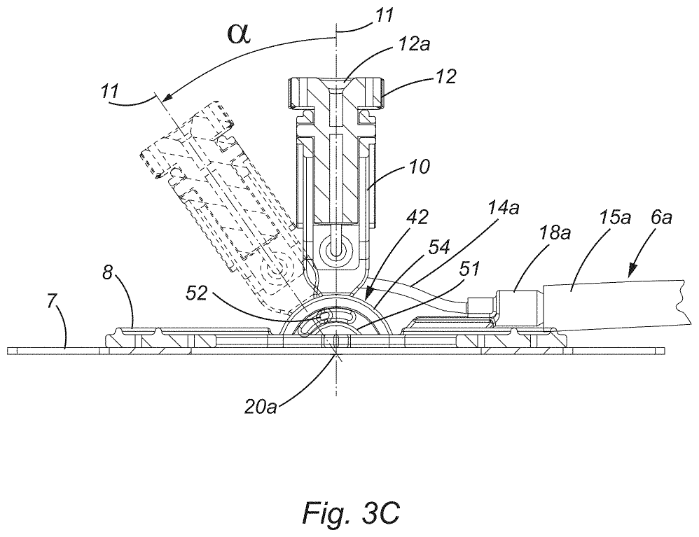

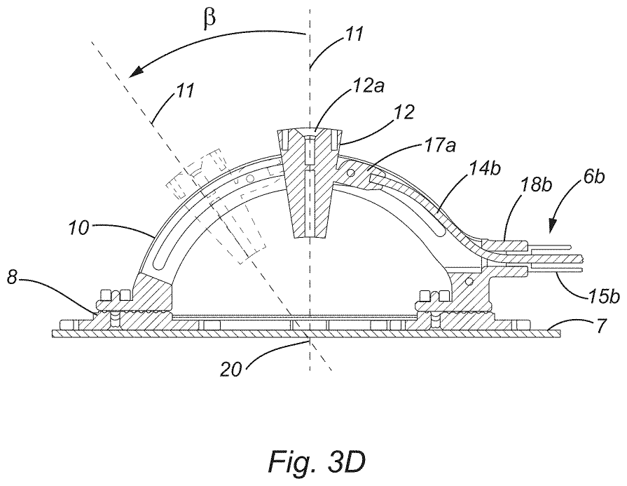

FIG. 3A shows a perspective view the guide assembly 2 according to one embodiment of the disclosure. FIG. 3B shows an exploded view of the components of guide assembly 2. FIGS. 3C and 3D show partial cross section views of the guide assembly.

Guide assembly 2 includes an adhesive patch 7 to removably affix the assembly to a patient's skin. As shown in FIG. 3A, one or more adhesive patches 7 are connected with the base 8. Patches 7 connect the guide assembly 2 with the skin of a patient being treated. According to one embodiment, a single continuous patch 7 is provided that consists of lobes or petal-shaped areas. The lobes of patch 7 allow the patch to conform to curved surfaces of a patient's body, for example, a patient's abdomen. According to other embodiments, patch 7 is continuous and does not have petal-shaped areas. According to one embodiment, patch 7 has a thickness less than about 0.5 mm. According to other embodiments patch 7 comprises a relatively thick layer of material, such as a foam, to allow the patch to better conform to the shape of the patient's body.

Patch 7 has a layer of pressure sensitive adhesive on its lower surface. The adhesive is a medically suitable adhesive for removably connecting devices to a patient's skin, for example, Medical Foam Tape 1773, Single Sided White Polyethylene, 83 #Liner manufactured by 3M Corp. The adhesive layer is provided with a removable cover layer. Once a patient has been prepared for a procedure, the protective layer is peeled from patch 7 to expose the adhesive layer. The practitioner positions guide assembly 2 so that an insertion point 20 is located where the physician intends to insert the needle or other instrument through the patient's skin. According to some embodiments, the protective layer may be partially peeled off from patch 7 so that device 2 can be temporarily positioned and repositioned. Once assembly 2 is in the correct position, the remainder of the protective layer of patch 7 is removed and patch 7 is pressed against the patient's skin to secure assembly 2 in place. According to some embodiments, patch 7 may have rigid molded elements to aid with stability of the device on the patient. According to another embodiment, instead of, or in addition to, a pressure sensitive adhesive layer, assembly 2 is secured using a suction mechanism such as a resilient suction cup or a chamber connected with a vacuum source such as an institutional suction line.

According to another embodiment, the removable protective layer has a lower surface that readily grips skin or other tissue. According to this embodiment, the protective layer remains intact while the practitioner adjusts the location of the assembly. The gripping surface holds assembly 2 in place temporarily until the practitioner is satisfied with the position and removes the protective layer to affix the assembly to the patient's skin.

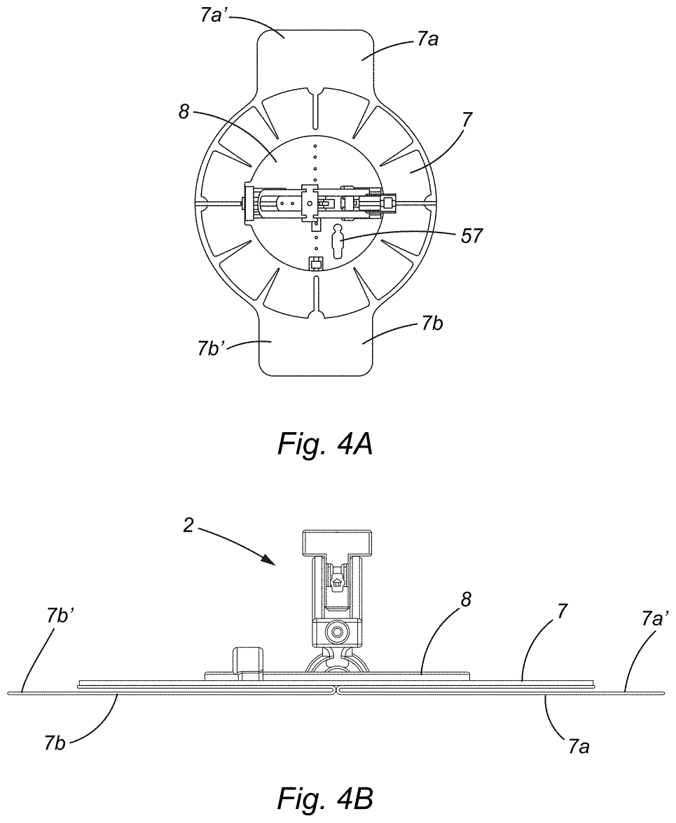

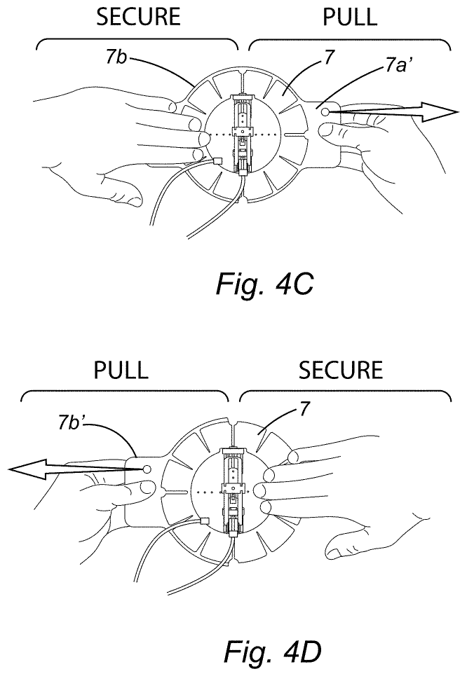

FIGS. 4A-D show an assembly 2 including patch 7 according to an embodiment of the disclosure. As shown in FIG. 4A, patch 7 is connected with the bottom of base 8. Peelable protective layers 7a and 7b are removably adhered to the bottom surface of patch 7. As shown in FIG. 4B, protective layers 7a, 7b have pull tabs 7a′ and 7b′ that extend from beneath assembly 2. Protective layers 7a, 7b extend from the pull tabs and fold back over themselves so that a portion of the protective layers contact, and are releasable adhered to, patch 7. This arrangement allows protective layers 7a, 7b to be removed from patch 7 by pulling respective pull tabs 7a′, 7b′. In the embodiments shown in FIGS. 4A-4D, protective layers 7a, 7b peel away from patch 7 from the center outward. The disclosure is not limited to peeling in this direction and encompasses a protective layer that peels away first from an edge of patch 7 towards the center of the patch.

As shown in FIG. 3A, base 8 is attached to the upper surface of patch 7. Base 8 has a central opening 9. According to some embodiments, insertion point 20 is at the geometric center of base 8 within opening 9. Insertion point 20 is the location at the plane of the patient's skin where a needle or other instrument inserted using the guide will pierce the skin of the patient. For some medical procedures, a surgical incision may be made at the insertion point 20 to facilitate insertion of the medical instrument. The insertion path 11 intersects this insertion point.

FIGS. 4C and 4D illustrate a method for adhering assembly 2 to the skin of a patient at a selected position and orientation. A practitioner identifies a point on the patient's skin where a device being guided by assembly 2 is to pierce the skin. The practitioner aligns insertion point 20 with the identified point on the patient's skin. According to one embodiment, the practitioner rotates assembly 2 so that it has a selected rotational orientation with respect to the patient's anatomy and/or to an axis of the imaging system 50. For example, in some applications, such as where a CT scanner is used assembly 2 may need to be oriented so that arch 10 is along the imaging plane of the imaging device 50. An indicator mark, such as mark 57 may be provided on an upward-facing surface of protective layer 7a, 7b, to assist the practitioner to identify the orientation of the device relative to the axial plane. Once assembly 2 is properly position, the practitioner secures the assembly in place by pressing on one side of patch 7 against the patient and removes protective layer 7a from a first side of the assembly by pulling on pull tab 7a′ as shown inf FIG. 4C. The exposed adhesive surface of patch 7 adheres assembly 2 to the patient. The practitioner then stabilizes assembly 2 by pressing on the first side and removes the second protective layer 7b by pulling on pull tab 7b′ as shown in FIG. 4D. The practitioner can then press each portion of patch 7 to the skin of the patient to assure that the assembly is firmly held in place. Lobes of patch 7 may be pressed individually against the patent's skin to conform to the shape of the patient's body.

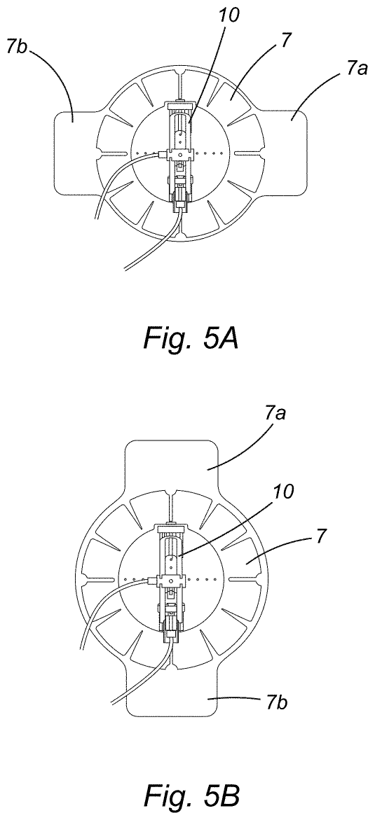

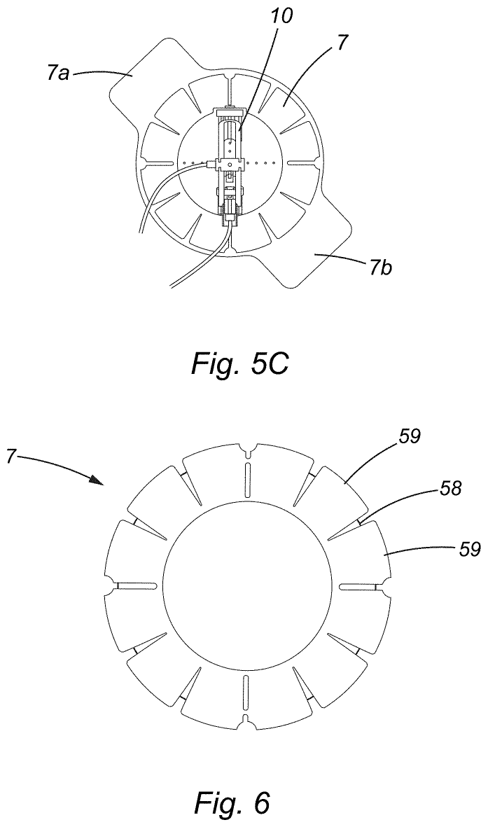

FIGS. 5A-5C show further embodiments of the disclosure. As shown in FIG. 5A, the orientation of protective layers 7a, 7b relative to assembly 2 is selected so that the protective layers separate along a plane aligned with arch 10. As shown in FIG. 5B, protective layers are arranged to separate along a plane perpendicular to the plane of arch 10. FIG. 5C shows the protective layers 7a, 7b arranged so that they separate along a plane at a selected angle relative to arch 10.

FIG. 6 shows another embodiment of patch 7. As with previous embodiments, patch 7 includes a plurality of lobes 59 that are flexible relative to assembly 2 so that patch can conform to curved portions of a patient's anatomy. Adjacent lobes 59 are connected to one another by breakable links 58. According to this embodiment, links 58 hold lobes 59 in a stable relationship with one another so that, when protective layers 7a, 7b are peeled away from patch 7, the configuration of patch 7 remains substantially flat and flexing between lobes 59 is reduced. This avoids having adhesive surfaces of the lobes 59 contact one another. Once the patch 7 is placed against the patient's skin and layers 7a, 7b are peeled away, the practitioner can press individual lobes 59 against the skin, and where necessary, break links 58 so that the lobes securely adhere to curved portions of the patient's anatomy.

As shown in FIGS. 3A-3C, arch 10 is connected at both ends with base 8 by hinges 42. According to a preferred embodiment, hinges 42 are sliding hinges, as will be explained below. Arch 10 extends along a semicircular path with a radius of curvature centered on insertion point 20. According to some embodiments, the diameter of arch 10, and thus the clearance between the arch and the patient's skin, is selected to provide sufficient space to allow a practitioner to reach the patient's skin at the insertion point 20 to make a surgical incision

Instrument guide body 12 is slideably connected with arch 10 so that it can slide along arch 10. Instrument guide body 12 includes bore 12a that defines an insertion path 11 of a medical instrument that slides through bore 12a. Bore 12a may include a coating for example, pertetrafluoroethylene (PTFE) that reduces friction with an instrument inserted through guide body 12 to provide the practitioner with an uninterrupted haptic sense of tissues being pierced by the needle or other medical instrument. Bore 12a is sized to closely match the outer diameter of the needle or other instrument to be guided by the apparatus so that the direction of motion of the instrument is closely aligned with the axis of bore 12a. According to some embodiments, instrument guide body 12 is provided with a motorized traction mechanism connected with bore 12a that moves a needle or other medical instrument along the insertion path 11. According to some embodiments, the traction mechanism allows a medical procedure to be performed robotically.

According to some embodiments, hinges 42 are slide hinges that position the axis of rotation 20a of arch 10 below the plane of base 8 and patch 7 and co-planar with the patient's skin. FIG. 3C shows a partial cross section view of guide assembly 2 viewed along the axis of rotation 20a of hinges 42. As shown in FIG. 3C, hinges 42 include support surface 51 connected with base 8. Support surface 51 is semicircular with a radius of curvature centered on an axis of rotation 20a. Axis of rotation 20a is in the same plane as insertion point 20, that is, at the surface of the patient's skin. Arch 10 includes slider 52 that rests on and slides along surface 51. Slider 52 has a lower curved surface that matches the curvature of surface 51. Arch 10 extends perpendicular from slider 52 so that the plane defined by arch 10 remains radially aligned with axis 20a as arch 10 rotates about axis of rotation 20a.

Retainer 54 extends from base 8 and is concentric with surface 51. The lower surface of retainer 54 contacts the upper surface of slider 52 so that slider 52 is captured between support surface 51 and retainer 54 and remains in sliding contact with surface 51. Support surface 51 may include one or more ridges to reduce the surface area of contact between surface 51 and slider 52 to reduce friction between the support surface and the slider. Adjustment of arch 10 about axis of rotation 20a changes the angle α, as shown in FIG. 3C

According to one embodiment, retainer 54 includes a slot 54a as can be seen in FIGS. 3A and 3B. Slider 52 includes one or more posts 52a that extend upward from slider 52. Posts 52a extend into slot 54a so that the slider 52 is prevented from moving axially with respect to support surface 51 and retainer 54. Posts 52a maintain the position of slider 52 in alignment with surface 51 and with base 8 to assure that arch 10 rotates about axis of rotation 20a and remains concentric with insertion point 20. According to other embodiments, instead of posts 54, snap engagements or molded features are provided on slider 52, support surface 51, and/or retainer 54 to maintain the slider in engagement with support surface and in alignment with base 8.

According to one embodiment, the contacting surfaces of support 51 and slider 52 of hinge 42 are selected to provide static friction to hold the orientation of arch 10 until force is applied. According to other embodiments, one or more of hinges 42 include a locking mechanism to set the angle of arch 10 with respect to ring 8. According to some embodiments, the lock mechanism includes a locking screw that releasably engages slider 52 and surface 51 to fix the orientation of arch 10 about axis of rotation 20a.

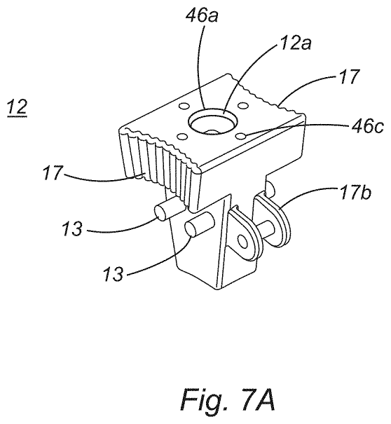

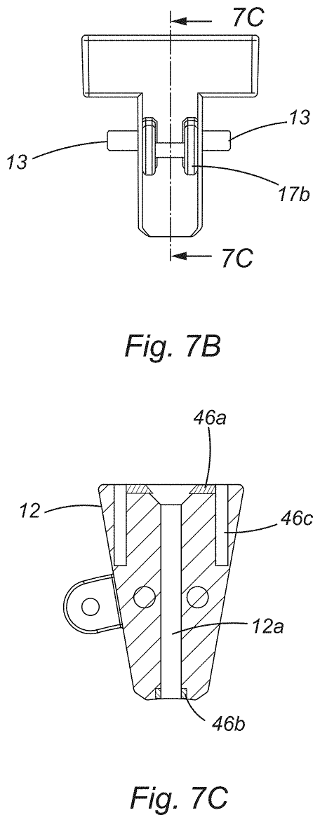

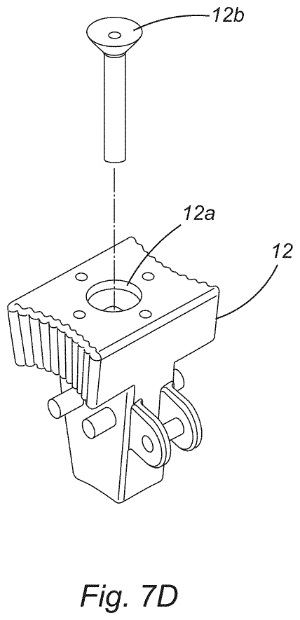

Guide body 12 is slideably positioned along arch 10. According to one embodiment, arch 10 includes segments 10a, 10b separated by a gap. Segments 10a, 10b each include a respective slots 16a, 16b. Guide body 12 is positioned in the gap between segments 10a, 10b. FIG. 7A shows a perspective view of guide body 12. Posts 13 extend from the sides of body 12. As shown in FIG. 7A, posts 13 engage with slots 16a, 16b of segments 10a, 10b of arch 10. According to one embodiment, two or more posts 13 are provided on the sides of body 12 that engage with slots 16a, 16b so that body 12 has a fixed radial orientation with respect to arch 10. Engagement between body 12 and segments 10a, 10b allows body 12 to slide along the segments while keeping the guide body at a fixed radial orientation with respect to arch 10. According to one embodiment, contacting surfaces of body 12 and segments 10a, 10b are selected to provide a small amount of static friction so that body 12 will maintain its position along arch 10 until force is applied to reposition the body. Guide body 12 may include one or more gripping surfaces 17 to allow a practitioner to grasp the guide body directly and reposition it along arch 10.

Introducer bore 12a is provided through guide body 12. Bore 12a is aligned with the radius of arch 10. Bore 12a is sized and shaped to conform to the outer surface of a medical instrument, such as an introducer, cannula, biopsy needle, and the like and sized so that the path of motion of the instrument extending through bore 12a remains co-linear with the axis of the bore.

According to some embodiments, bore 12a can be adjusted by adding or removing a cylindrical insert 12b that conforms to the inner diameter of the bore and have an inner diameter that conforms to a particular instrument. For example, bore 12a may have an inner diameter sized to accommodate a 14-gauge biopsy needle (i.e., a diameter of about 2.1 mm). As shown in FIG. 7D, a removable insert 12b is fitted into bore 12a. Insert 12b has an inner diameter sized to fit an instrument with a smaller diameter, for example, a 22-gauge spinal needle with a diameter of about 0.72 mm. When insert 12b is fitted into bore 12a, the smaller-diameter instrument can be guided by assembly 2. A range of inserts 12b can be provided according to embodiments of the disclosure allowing guide assembly 2 to be modified to guide the insertion of a variety of medical instruments. The length of bore 12a and of insert 12b is selected so that the range of deviation of the tip of the instrument is limited, regardless of the diameter of the instrument.

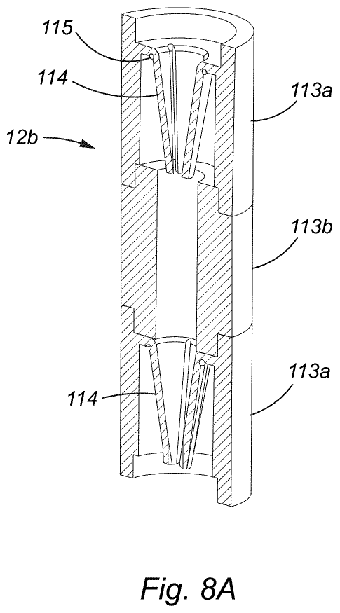

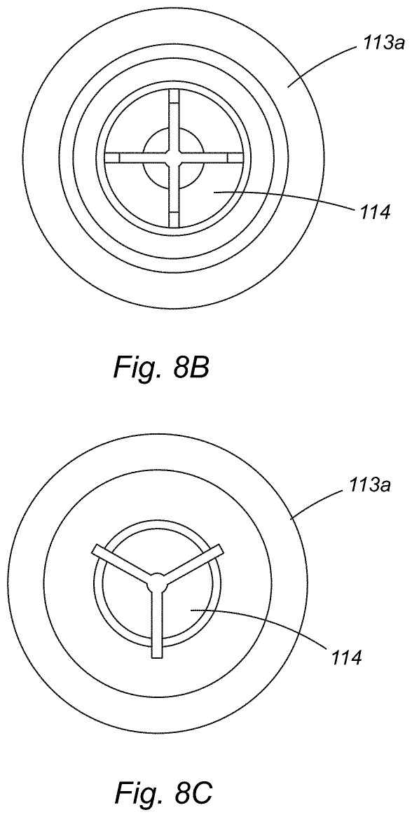

FIGS. 8A-8C show a detailed view of cylindrical insert 12b. According to one embodiment, insert 12b is removably disposed bore 12a of guide 12. According to another embodiment, insert 12b is fixed within bore 12a. According to a still further embodiment, insert 12b in FIGS. 8A-8C is integral with guide body 12, formed, for example, when guide body 12 is molded.

According to one embodiment, insert 12b is formed by a plurality of membranes arranged along insertion path 11 and adapted to guide an instrument inserted along bore 12a into alignment with the insertion path. In the embodiment shown in FIGS. 8A, 8B, and 8C, the membranes comprise top and bottom funnel sections 113a and a center section 113b. Funnel sections 113a have a plurality of tines 114 arranged about the central axis of the insert 12b. The inward slope of the tines 114 directs the instrument toward the central axis. Tines 114 slope inward so that the bottom-most tips of the tines define an opening smaller than the diameter of the instrument. Preferably, the opening defined by the ends of tines 114 is less than about 0.7 mm. Tines 114 flex outward from the central axis when a needle or other instrument is inserted through insert 12b, widening the opening at the ends of tines 114. According to a preferred embodiment, tines flex outward to allow passage or instruments with a diameter greater than about 5 mm.

Tines 114 may be made from a material that has a relatively low modulus of elasticity to allow sufficient flexibility for the instrument to pass through the bore, while also having sufficient stiffness that the tines 114 make sliding contact with the instrument and hold the instrument along the axis of bore 12a. The inward sloping shape of the tines guide the instrument along the central axis of insert 12b. An undercut 115 may be provided where each of the tines 114 joins the body of funnel section 113a to modify the flexural modulus of the tines to adjust the friction the instrument will encounter as it passes along the insertion axis 11.

The two funnel sections 113a hold the instrument colinear with the central axis of insert 12b, and therefore, colinear with insertion axis 11, as discussed above. Central section 113b separates the upper and lower funnel sections 113a. Center section 113b has a central opening wide enough to allow passage of the instrument. The length of central section 113b may be selected to assure that the funnel sections 113a exert sufficient leverage on the instrument to hold it colinear with the insertion axis. Funnel sections 113a and central section 113b are shaped to stack together as shown in FIG. 8A.

Funnel sections 113a may have three or more tines 114 arranged along their central axis. As shown in FIG. 8B shows a funnel section 113a with four tines 114. FIG. 8C shows a funnel section 113a with three tines 114. The disclosure is not limited to three or four tines and includes inserts 12b with a fewer or a greater number of tines 114.

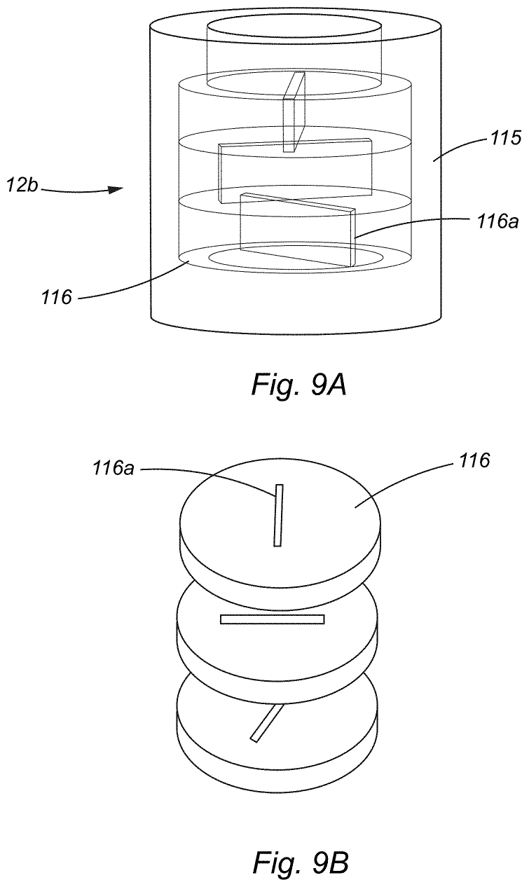

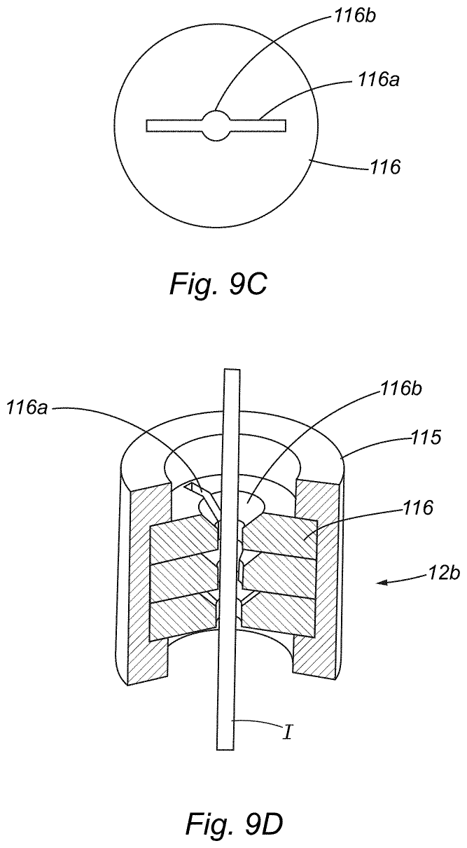

FIGS. 9A-9D show inserts 12b according to other embodiments of the disclosure. As shown in FIGS. 9A and 9B, insert 12b has an outer housing 115. Housing 115 may be a structure formed separately from guide body 12 and inserted into bore 12a. Alternatively, housing 115 is a portion of guide body 12 surrounding bore 12a. Arranged within housing 115 are a plurality of guide membranes 116. Each membrane 116 has a slit 116a that crosses a central point of the membrane. Membranes 116 are stacked within housing 115 with their respective slits 116a arranged in different rotational orientations. Because each slit crosses the central point of membrane 116, the slits 116a together define an opening along the central axis of insert 12b. Membranes are formed from a material that is rigid enough to prevent piercing by the instrument while also being malleable enough to flex around the outer diameter of the instrument and force the instrument toward the central axis.

FIG. 9C shows a membrane 116 according to another embodiment of the disclosure.

Slit 116a includes a central circular opening 116b at the midpoint of slit 116a and at the central point of membrane 116. The size of opening 116b is selected to correspond to the outer diameter of an instrument inserted along insertion axis 11. Opening 116b may be smaller than the diameter of the instrument to provide an interference fit to assure that the instrument remains aligned with the central axis of guide 12.

FIG. 9D shows a cross section of an insert 12b including a stack of membranes 116 according to another embodiment of the disclosure. Central opening 116b in this embodiment includes a sloped surface to direct an instrument, I, inserted into guide body 12 toward the center of the membrane to align the instrument with the insertion axis 11.

As shown in FIG. 3D, guide body 12 slides along arch 10 to adjust an angle of insertion path 11 along the plane defined by arch 10. As the position of needle guide 12 is adjusted along arch 10, insertion path 11 is adjusted. Because insertion point 20 is at the radial center of arch 10, insertion path 11 intersects insertion point 20, regardless of the position of guide body 12 along arch 10. Adjustment of the position of guide body 12 along arch 10 changes the angle β of insertion path 11 as shown in FIG. 3D.

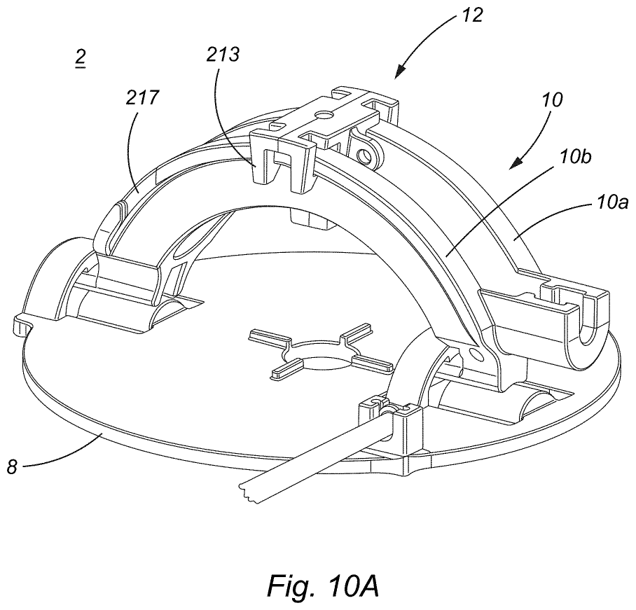

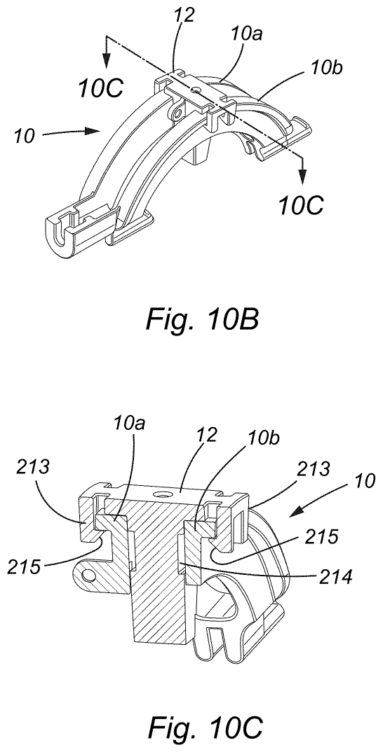

FIGS. 10A-10C show assembly 2 including an arch 10 and guide body 12 according to another embodiment of the disclosure. Arch 10 is formed from rails 10a, 10b. Guide body 12 includes arms 213. Rails 10a, 10b are shaped to fit within openings formed beneath arms 213. As shown in the cross section in FIG. 10C, arms engage with rails 10a, 10b. Arms 213 extend below the underside of rails 10a, 10b to hold guide body 12 against the rails. The dimensions of rails 10a, 10b and arms 213 are selected to allow guide body 12 to slide smoothly along rails 10a, 10b. According to one embodiment, cutouts 214 are provided on guide body 12 to reduce the contact area between guide body 12 and rails 10a, 10b to further reduce friction as guide body 12 moves long rails 10a, 10b.

According to one embodiment, arms 213 include chamfers 215. Arms 213 are formed from a material sufficiently flexible so that arms 213 can flex outward from guide body 12. According to this embodiment, during manufacturing of assembly 2, guide body 12 is engaged with arch 10 by pressing body 12 between arms 10a, 10b so that chamfers 215 ride on the edges of the rails, driving arms 213 away from guide body 12 until the ends of the chamfers pass the edges of the rails. Resiliency of the material forming arms 213 causes the arm to rebound, so that guide body 12 snaps into place on arch 10.

According to another embodiment, rails 10a, 10b each include a cut-away portion 217. Cut away portions 217 allow guide body 12 to fit into the gap between rails 10a, 10b without having to flex arms 213 away from body 12. Instead, chamfers 215 of guide body 12 slide through cut away portions 217 and guide body 12 is moved upward along arch 10 so that rails 10a, 10b fit within the space formed by arms 213. According to one embodiment, cut away portions 217 are shaped so that guide body 12 is inserted with bore 12a oriented vertically in the orientation shown in FIG. 10A. Once the chamfers 215 on one side of body 12 are below rails 10a, 10b, body 12 is rotated so that the opening below arms 213 is aligned with the rails 10a, 10b. Body 12 is then moved proximally along rails so that rails 10a, 10b extend fully through the space beneath arms 213.

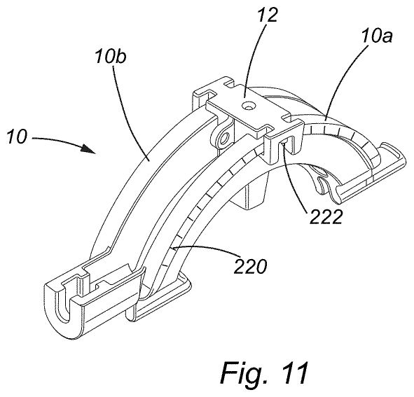

FIG. 11 shows arch 10 according to a further embodiment of the disclosure. Markings 220 are provided along one side, or along both sides of rails 10a, 10b. Markings 220 are arranged at regular angular intervals along arch 10, for example, every 5 or 10 degrees. Guide body 12 includes an opening 222 adjacent to the markings 220. In use, a practitioner views markings 220 visible through opening 222 to observe the angular position of guide body 12, and hence the orientation of insertion axis 11.

As shown in FIG. 2, guide assembly 2 is connected with remote operators 4a and 4b by cables 6a and 6b, respectively. As shown in FIG. 3C, cable 6a adjusts the orientation of arch 10 about axis of rotation 20a. According to one embodiment, cable 6a is a Bowden cable consisting of an inner shaft 14a surrounded by a sheath 15a. The distal end of sheath 15a is fixed with base plate 8. Strain relief 18a may be provided on base 8 to receive the distal end of sheath 15a. Shaft 14a extends through strain relief 18a and is connected with arch 10. Shaft 14a is movable in the proximal and distal directions within sheath 15a. A lubricious coating or a low-friction material such as PTFE may be provided between shaft 14a and sheath 15a to provide ease of motion. Motion of shaft 14a is communicated to arch 10 to adjust the angle α of the arch. According to one embodiment, the angle α can be adjusted between about −50° and +60°. According to a preferred embodiment, the angle α can be adjusted between about −70° and +70°. According to a most preferred embodiment, the angle α can be adjusted between about −80° and +80°.

Cable 6b adjusts the position of needle guide 12 along arch 10. As shown in FIG. 3D, cable 6b includes outer sheath 15b and inner shaft 14b. Strain relief 18b may be provided on arch 10. Sheath 15b is connected with strain relief 18b. Shaft 14b extends from sheath 15b, through strain relief 18b, and extends partially along arch 10. The distal end of shaft 14b is connected with guide body 12.

According to one embodiment, the distal end of shaft 14b is connected directly with guide body 12. According to another embodiment, the distal end of shaft 14b is connected with guide body 12 by hinge components 17a, 17b. According to one embodiment, first hinge component 17a is connected with the distal end of shaft 14b. First hinge component 17a couples with second hinge component 17b on guide body 12, as shown in FIG. 7A. According to this embodiment, hinge components 17a and 17b are engaged by an axle through both components. This arrangement limits the movement of the end of shaft 14b so that it remains in the plane defined by arch 10 and communicates force applied by shaft 14b onto guide body 12 to drive it along arch 10.

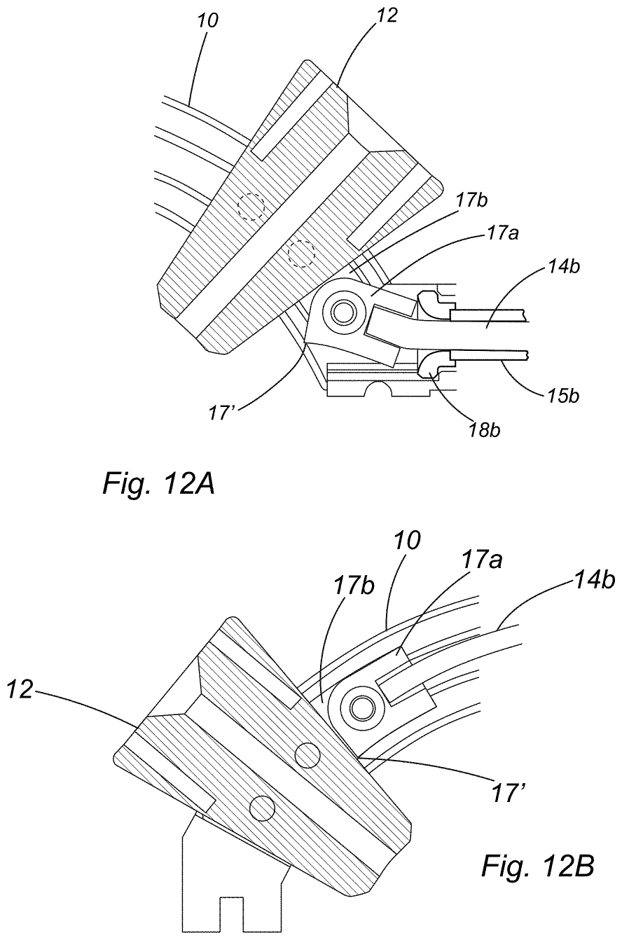

FIGS. 12A and 12B are cross sections showing a detained view of the engagement of first and second hinge components 17a, 17b according to an embodiment of the disclosure. Component 17a is affixed to the end of shaft 14b. FIG. 12A shows guide body 12 positioned at the proximal end of arch 10. Hinge component 17a is titled slightly upward with tab 17a′ tilted away from the surface of body 20. Force applied by the extension of shaft 14b causes body 12 to move in the distal direction along arch 10. As guide body 12 moves, hinge component 17a rotates clockwise with respect to guide body 12. When guide body 12 reaches the distal end of arch 10, hinge component 17a has rotated so that tab 17a′ contacts the surface of guide body 20. Force applied to body 20 by tab 17a′ allows body 20 to be pushed to the distal-most end of arch 10 without kinking shaft 14b.

As shaft 14b moves proximally and distally with respect to sheath 15b, needle guide 12 is moved along arch 10 to adjust the angle β of the arch, as shown in FIG. 3D. According to one embodiment, the angle β can be adjusted between about −45° and +45°. According to a preferred embodiment, the angle β can be adjusted between about −60° and +60°. According to a most preferred embodiment, the angle β can be adjusted between about −80° and +80°.

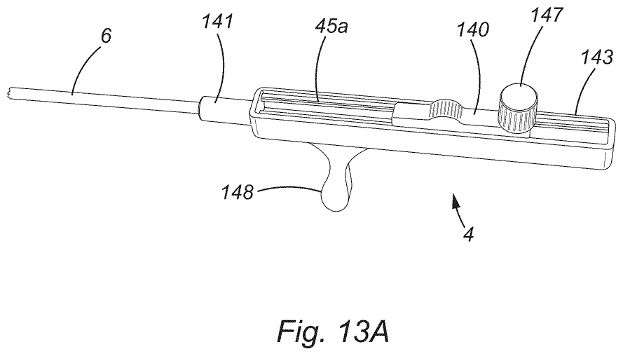

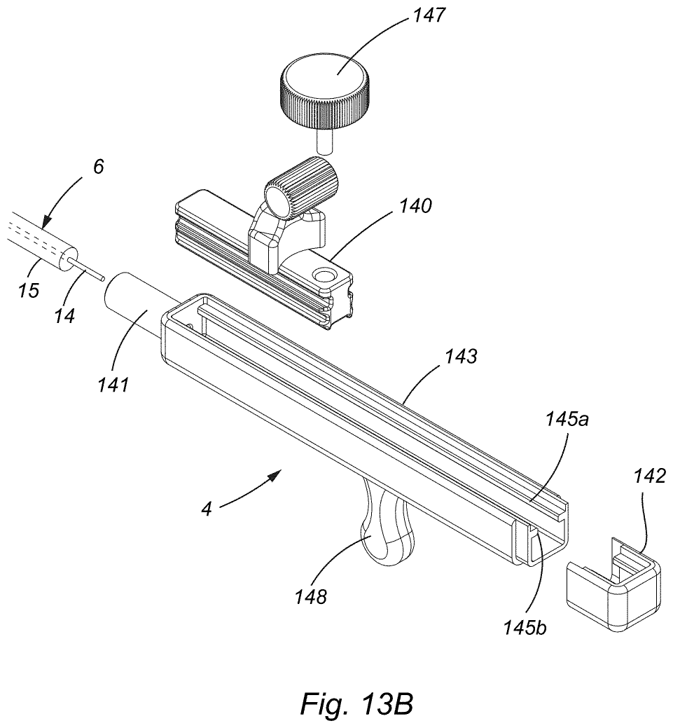

FIG. 13A shows a perspective view of remote operator 4 according to an embodiment of the disclosure. FIG. 13B shows an exploded view of the remote operator 4. As show in FIG. 2, the same design of the remote operator 4 is connected with each of cables 6a and 6b. Portions of the operators 4 may have distinguishing features, such as colors, labeling, embossments, and the like, to allow the operator to readily distinguish which operator drives the arch 10 to adjust angle α and which drives the guide body 12 to adjust angle β.

According to a further embodiment, rolling or twisting actuators are provided in place of, or in addition to, linear displacement of a sliding actuator. In this embodiment, proximal ends of shafts 14a, 14b engage with spindles or lever arms within the operator. A practitioner applies rotational motion to the spindle or lever arm. Winding of shaft 14a, 14b around spindle or displacement of the shaft by motion of the lever arm displaces the shaft proximally and distally to change the orientation of arch 10 and guide body 12.

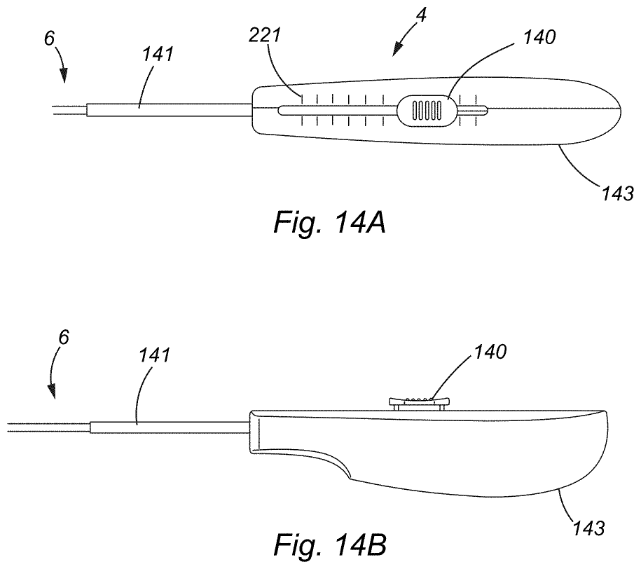

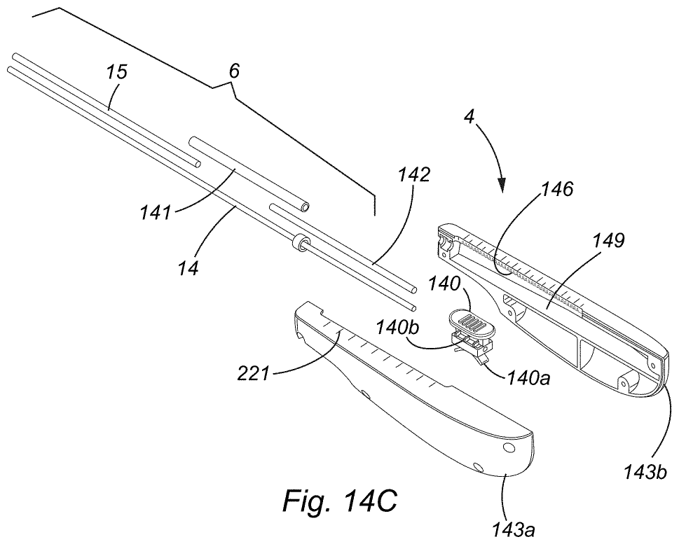

According to another embodiment, actuator 140 is formed by a threaded rod that engages with a corresponding internal thread on housing 143. The distal end of the rod is connected with the proximal end of a corresponding shaft 14a, 14b. A knob is provided on the proximal end of the threaded rod. Corresponding sheath 15a, 15b is fixed with the housing 143. To change the orientation of insertion path 11, the practitioner rotates the threaded rod relative to the housing by turning the knob, displacing the threaded rod along the internal thread of housing 143, and moving the shaft 14a, 14b proximally and distally to change the orientation of arch 10 and/or guide body 12. Such an embodiment allows the practitioner to make fine adjustments to the orientation of insertion path 11. According to a still further embodiment, a threaded rod adjustment is provided in combination with a sliding adjustment mechanism, such as shown by FIGS. 13A to 14C. According to this embodiment, a practitioner can make crude adjustments to the orientation of insertion path 11 using the sliding mechanism and then make fine adjustments by rotating the threaded rod.

Cable 6 (i.e., cable 6a or 6b shown in FIG. 2) is connected with the distal end of operator 4. According to one embodiment, strain relief 141 connects cable 6 with operator 4. Strain relief 141 is fixed with the outer sheath (i.e., 15a, 15b) of the cable. Inner shaft 14a, 14b extends through strain relief 141 and into the interior of housing 143. Sliding actuator 140 is provided in housing 143. As shown in the exploded view of FIG. 13B, rails 145a, 145b are provided on the inside surfaces of housing 143. Actuator 140 slides along the rails 145a, 145b in the proximal and distal directions. The proximal end of the shaft 14a, 14b of the respective cable 6a, 6b is connected with actuator 140. Motion of actuator 140 proximally and distally within housing 143 moves the shaft relative to the cable sheath. Because the sheaths 15a, 15b are fix to the base 8 and arch 10, respectively, motion of shafts 14a, 14b is communicated to the arch 10 and guide body 12 of the guide assembly 2. According to some embodiments, sliding actuator 140 may have different ranges of motions between the remote operator 4a, used to adjust the orientation of arch 10 and remote operator 4b, used to adjust the position of guide body 12 along arch 10. According to one embodiment, the range of motion of the actuator 140 of remote operator 4a is less than that of remote operator 4b. The remote operators 4a, 4b may be of different sizes or may be of the same size.

According to some embodiments, locking knob 147 may be provided on actuator 140. Knob 147 may include a threaded engagement so that turning the knob in one direction fixes the actuator 140 to the housing 143, thus fixing the position of the respective guide body 12 or arch 10 and hence, the angles α and β of the insertion path 11.

As shown in FIG. 13B, housing 143 may be provided with an end piece 142. During assembly, end piece 142 is disconnected from housing 143 to allow actuator 140 to be inserted in the housing along rails 145a, 145b. End piece 142 is then connected with housing 143 to keep actuator captive within housing 143. According to some embodiments, end piece 142 has a snap-fit engagement with housing 143.

According to some embodiments, a grip 148 is provided on the bottom of housing 143. Grip 148 allows the operator to comfortably hold the operator 4 and the move actuator 140 with one hand. This allows a practitioner to adjust the insertion path 11 by controlling the position of the arch 10 and guide body 12 with the practitioner's right and left hand, respectively.

According to some embodiments, operators 4 are shaped be operated by either the left or right hand (i.e., they are ambidextrous). Portions of the operator, for example, the actuator 140, may be formed from different colored material with the operator 4a that adjusts the angle of arch 10, α, colored grey and the operator 4b that adjusts the angle of the guide body 12, β, colored blue. According to other embodiments, the operators 4 are shaped so that one is comfortably operated by the left hand and the other by the right hand. Such an arrangement may be advantageous to prevent operator confusion regarding which angle α or β of the insertion path is being adjusted.

According to some embodiments, instead of two separate remote operators 4a, 4b, the sliding mechanisms to adjust the position of arch 10 and guide body 12 are combined into a single housing. According to some embodiments, the housing for the combined mechanisms is shaped to allow the practitioner to adjust the arch 10 and guide body 12 orientations using one hand.

FIGS. 14A-14C show remote operator 4 according to another embodiment of the disclosure. Housing 143 is shaped to be easily and comfortably grasped by a practitioner. Actuator 140 extends from the top surface of housing 143 and is slidable in the proximal and distal directions. Actuator 140 includes a locking mechanism that holds the actuator 140 fixed with respect to housing 143, hence holding the position of arch 10 or guide body 12 fixed with respect to base 8 of assembly 2. Actuator 140 is unlocked by pressing downward. This allows actuator 140 to be moved relative to housing 143 to reposition the arch 10 or guide body 12.

FIG. 14C shows an exploded view of remote operator 4 according to this embodiment. Housing 143 is formed by two halves 143a, 143b. Each half includes a slide surface 149 extending along the length of the housing 143. Positioned on the underside of the top surface of housing halves 143a, 143b is a toothed rail 146. Actuator 140 includes springs 140a extending downward and ridges 140b extending upward. When halves 143a, 143b are joined, actuator 140 is held within housing 143 with springs 140a pressed against slide surface 149. Resiliency of springs 140a press ridges 140b against toothed rails 146. Engagement of ridges 40b and toothed rails 146, locking actuator 140 from moving relative to housing 143. Pressing downward on actuator 140 compresses springs 140a, disengaging ridges 140b from toothed rails 146, allowing actuator 140 to move proximally and distally relative to housing 143. As with previous embodiments, inner shaft 14a or 14b of cables 6a or 6b is fixed to actuator 140 and outer sheaths 15a, 15b are fixed to housing 143. According to one embodiment, strain relief 141 surrounds the cables 6a, 6b to avoid kinking of the cables where they connect with the housing. According to one embodiment, anti-kinking support 142 is provided around the proximal end of shafts 14a, 15a within housing 43. Anti-kinking support 142 slides inside strain relief 141 as actuator 140 moves relative to housing 143.

According to some embodiments, markings 221 are provided on the housing 143 and/or the actuator 140 that show the position of the actuator along the length of the housing. These marking may be calibrated to correspond to the angular orientations, α and β of the insertion path 11.

According to one embodiment, actuator 140 and/or housing 143 include a mechanism that provides an audible or tactile sensation as the actuator is moved proximally and distally. According to one embodiment, mutually engaging features on the actuator 140 and housing 143 flex as they engage one another, generating an audible “click” and/or a vibration of the operator 4a, 4b at regular intervals corresponding to the angular displacement of the arch 10 and guide body 12, for example, every 5 or 10 degrees of displacement. This mechanism may comprise features on toothed rail 146 and ridges 140b that partially engage when actuator 140 is pressed downward. For example, toothed rail 146 may include regularly spaced flexible extensions that flex and slide over ridges 140b to generate a vibration and/or clicking sound. This embodiment provides a practitioner with audible and tactile feedback about the changes in orientation of insertion path 11.

As shown in FIG. 1, according to some embodiments, the length of cables 6a and 6b is select to allow a practitioner to adjust the orientation of the guide assembly 2 from a distance away from the imaging system 50. According to some embodiments, the length of cables 6a, 6b is greater than about 1000 mm. According to another embodiment, the length of cables 6a, 6b is between about 30 centimeters (cm) and 1000 cm. More preferably, the length of cables 6a, 6b is between about 30 cm and 250 cm. Most preferably, the length of cables 6a, 6b is about 140 cm. The length of cables 6a, 6b may be selected to allow a practitioner to operate the device while remaining at a safe distance from ionizing radiation emitted by imaging device 50. The lengths of cables 6a, 6b may also be selected so that during normal operation they do not hang down so low as to reach the floor to avoid contamination. Cables 6a, 6b may be formed from flexible materials, for example, PTFE, so that inadvertent motion of the operators 4a, 4b is not communicated to the guide assembly 2. This reduced the risk that motion of the practitioner's hands will dislodge the guide assembly from its selected position on the patient. Cables 6a, 6b may include hook-and-loop fasteners, adhesive tape, magnetic clips, or the like to removably hold any excess length of the cables in a coiled configuration so that the cables do not interfere with other apparatus, for example, the motorized bed of imaging device 50 or hang down and touch the floor.

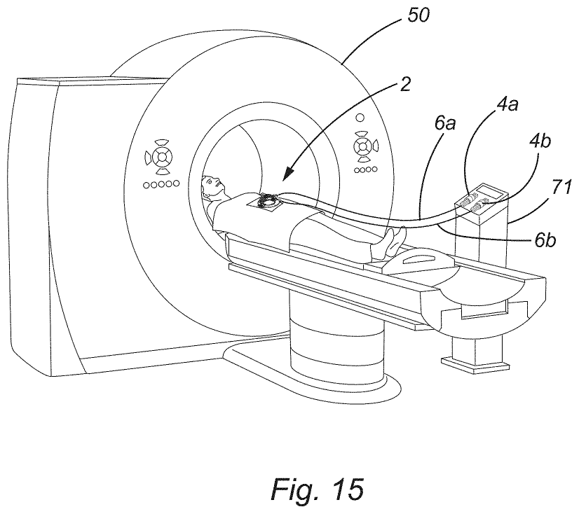

According to a further embodiment shown in FIG. 15, a motorized operator 71 is provided. Motorized operator 71 includes a mechanism for operating the actuators 140 of each of the operators 4a, 4b to adjust the orientation of the insertion path 11. Motorized operator 71 may comprise a communication system, such as a BlueTooth® link, that allows a practitioner to send commands to adjust the insertion path remotely. According to other embodiments, motorized operator 71 comprises a robotic system that adjusts the insertion path 11 in response to a computer program. In conjunction with motorized operator 71, guide body 12 may include a drive system for advancing and retracting a medical instrument through bore 12a and along insertion path 11. Signals from the motorized operator 71 or communicate directly to the guide body 12 could be used to perform a medical procedure.

According to some embodiments, the components of guide assembly 2 are formed from materials that are radio-transparent, that is, that have a low radio-opacity. This allows the practitioner to visualize features of the patient's tissue using a CT scan or fluoroscope without the guide assembly 2 obstructing the image of the tissue.

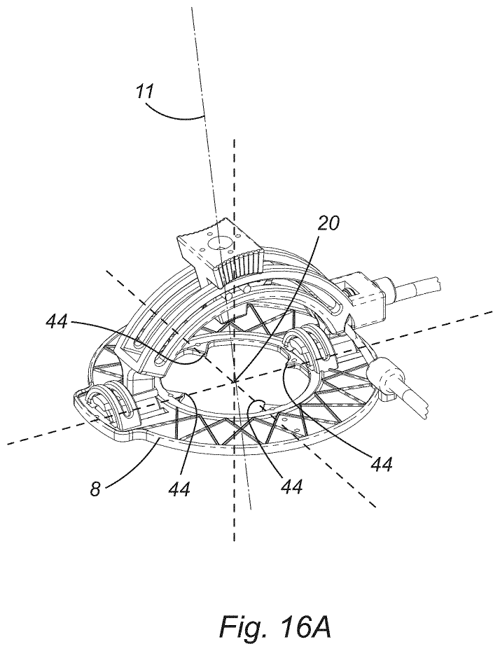

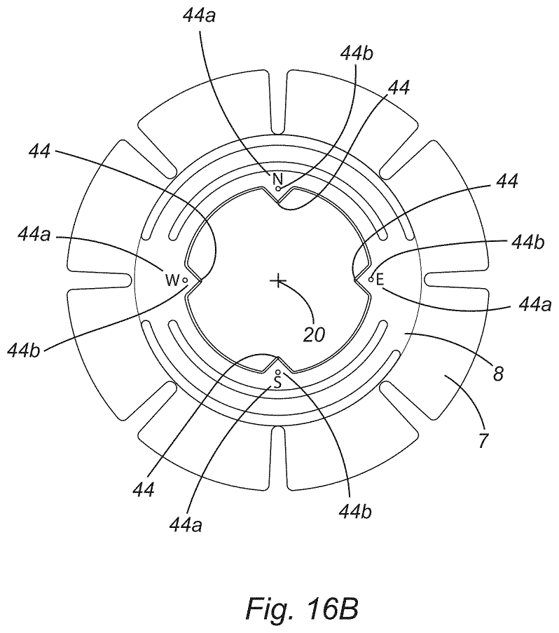

As shown in FIG. 16A, in order to facilitate positioning and orienting guide assembly 2 while the tissues are being imaged, one or more radiographic center alignment features 44 are provided on base 8. These may be radio-opaque, or at least have a radio-opacity greater than the other components of the guide assembly so that they are visible on imaging device 50. As shown in FIG. 16A, center alignment features 44 may be shaped as points or arrows directed toward the central insertion point 20. The practitioner can observe both the tissue to be treated and the guide assembly using the imaging system 50 and use the center alignment features 44 to assure that the guide is centered over the selected insertion point. While the targeted tissue and assembly 2, including features 44, are visualized, assembly 2 is repositioned until features 44 indicate that the assembly is aligned with insertion point 20. As discussed above, some or all of the protective layer covering the adhesive on patch 7 can then be removed and the patch pressed against the patient's skin to fix the position of assembly 2 relative to the patient's tissues.

Center alignment features 44 may also include radio-opaque structures that indicate the rotational orientation of guide assembly 2. FIG. 16B shows a base 8 according to a further embodiment. For clarity, the arch and other structures are not included in this figure. According to one embodiment, each feature 44 includes a distinct radio-opaque marking 44a such as a “compass” direction E, S, W, N. This allows the practitioner to see the radial orientation of the guide assembly 2 when viewed under x-ray imaging and to adjust the radial orientation of the guide assembly 2 before adhering patch 7 to the patient's skin. According to other embodiments, instead of, or in addition to, markings 44a that provide a two-dimensional image, markings 44a include three-dimensional features, such as cubes, spheres, or other shapes. Such an arrangement would enable markings 44a to be distinguished from one another on lateral, anteroposterior (AP), and 3-dimensional scans. The embodiment in FIG. 16B shows four center alignment features 44. Greater or fewer features could be provided with the scope of the disclosure.

Instead of, or in addition to, radio-opaque base alignment features 44, 44a optical alignment features 44b may be used. Some imaging systems 50 include a laser alignment system that projects a visual image onto the patient that identifies the center of the imaging field. According to one embodiment, features 44b are provided that are readily visible when the guide assembly is illuminated by a laser alignment system. In the embodiment shown in FIG. 16B, a series of holes 44b are provided. Laser light is reflected from the surface of the assembly while the holes do not reflect the light, thus providing a high-contrast indicator where the laser beam crosses. The holes may be arranged along lines that are co-linear with center alignment features 44. According to other embodiments, in place of, or in addition to holes 44b, other features such as reflective paint, reflective or holographic stickers, etched surfaces, and the like may be provided to show the position and orientation of the guide assembly 2 when illuminated by a laser alignment system.

As shown in the cross section of guide body 12 in FIG. 7C, one or more radiographic path alignment features 46a, 46b may be provided on guide body 12. Guide body 12 may be formed from a material with a low radio-opacity. A radio-opaque upper path alignment feature 46a is provided on guide body 12 at the top end of bore 12a. According to one embodiment, upper path alignment feature 46a has an annular ring encircling the axis of bore 12a. A lower path alignment feature 46b is located at the lower end of bore 12a and likewise encircles the axis of the bore. As will be described below, the practitioner uses the path alignment features 46a and 46b to visualize the insertion path. When the annular rings of 46a and 46b are concentric and centered on the tissue to be treated, the practitioner is assured that bore 12a, and hence, the insertion path 11, is aligned with the selected tissue. The embodiments described here include two path alignment features, but a greater or fewer number of path alignment features could be provided within the scope of the disclosure.

According to a further embodiment, path alignment feature 46a and/or 46b are shaped differently from one another to allow the practitioner to readily distinguish the upper feature 46a from the lower feature 46b when observing the guide using the imaging device 50. For example, upper feature 46a could have a square outline surrounding a central annular ring while lower feature 46b has a round outline. Differently shaped outlines allow the practitioner to distinguish the feature at the top of bore 12a from the feature at the bottom of the bore when the insertion path is being visualized under x-ray imaging.

Guide body 12 may include visual features that facilitate positioning when used with a laser alignment system. These features may include holes, reflective paint, reflective or holographic stickers, etched surfaces, and the like that interact with the laser projection system to allow the practitioner to visualize the orientation of guide body 12 with respect to the axis of the imaging system. In the embodiments shown in FIGS. 7A, 7B, and 7C, holes 46c are provided in the top surface of the body. These holes provide a stark contrast when the surface of body 12 is illuminated by the laser projection system.

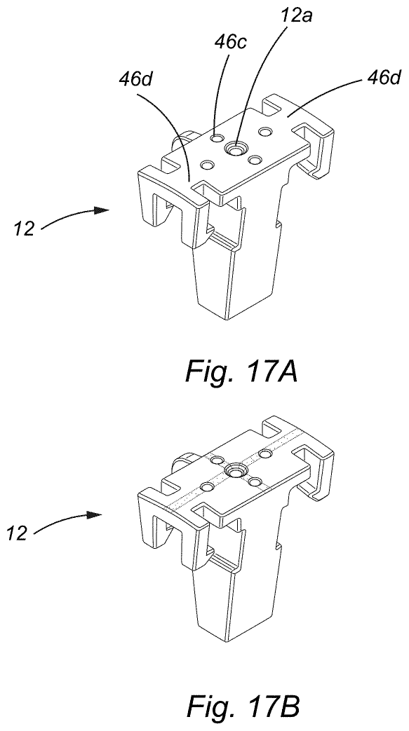

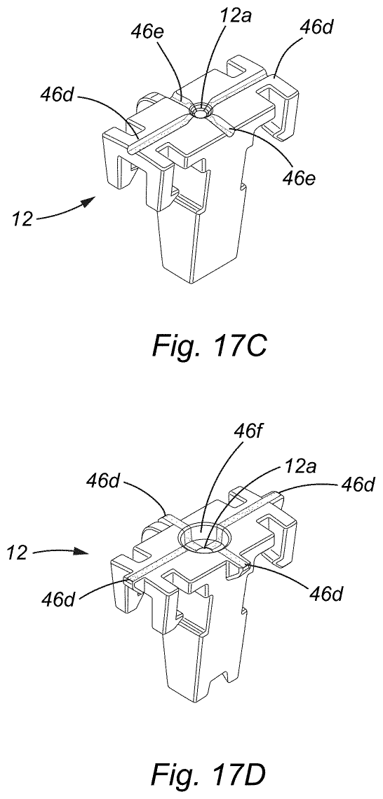

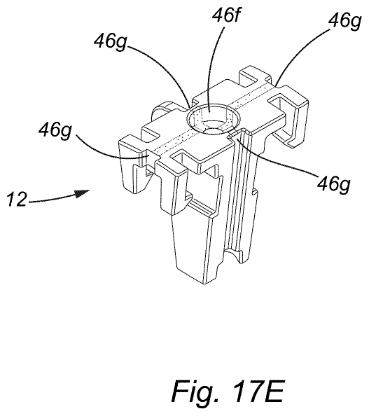

FIGS. 17A-17E show guide body 12 including laser alignment features according to further embodiments of the disclosure. In the embodiment shown in FIGS. 17A and 17B, the top surface of guide body 12 includes a plurality of holes 46c arranged in relation to bore 12a. The top surface includes narrowed extensions 46d on opposite sides of bore 12a. As shown in FIG. 16B, when the beam of an alignment laser of imaging system 50 illuminates guide body 12, the beam is scattered from the surface to create a bright image across the top surface and along extensions 46d. Holes 46c and bore 12a do not scatter the beam, resulting in high contrast dark features that allow a practitioner to confirm that the position of guide body 12 is aligned with the imaging system 50.

In the embodiment shown in FIG. 17C, instead of holes 46c, a plurality of grooves or slots 46e are provided in the top surface of guide body 12. Slots 46e are aligned with bore 12a and extend parallel to, and perpendicular to, the direction of travel of guide body 12 along arch 10. When guide body 12 is aligned with the imaging system, the beam of the laser alignment system falls along slots 46e. A reflective, or antireflective coating may be applied to the slots 46e to enhance the visibility of the slots when it is illuminated by the laser beam.

In the embodiment shown in FIG. 17D, four extensions 46d extend from the sides of guide body 12. A cavity 46f is formed around bore 12a. Extensions 46d are aligned with bore 12a and extend parallel to, and perpendicular to, the direction of travel of guide body 12 along arch 10. When guide body 12 is aligned with the imaging system, the beam of the laser alignment system falls along extensions 46d and also into cavity 46f, providing a practitioner with confirmation that the guide body 12 is aligned with the imaging system 50. A reflective, or antireflective coating may be applied to the extensions 46d and/or to cavity 46f to enhance the visibility of the extensions and the crater when it is illuminated by the laser beam.

In the embodiment shown in FIG. 17E, four notches 46f are provided along the sides of guide body 12. A crater 46e is formed around bore 12a. Notches 46f are aligned with bore 12a and extend parallel to, and perpendicular to, the direction of travel of guide body 12 along arch 10. When guide body 12 is aligned with the imaging system, the beam of the laser alignment system falls into notches 46f and also into crater 46e, providing a practitioner with confirmation that the guide body 12 is aligned with the imaging system 50.

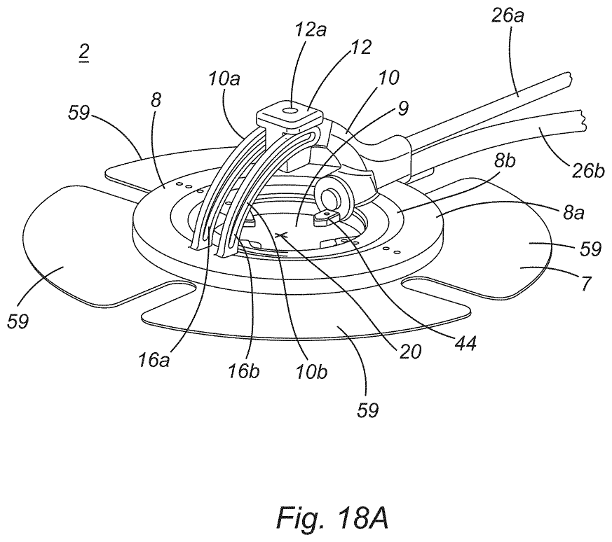

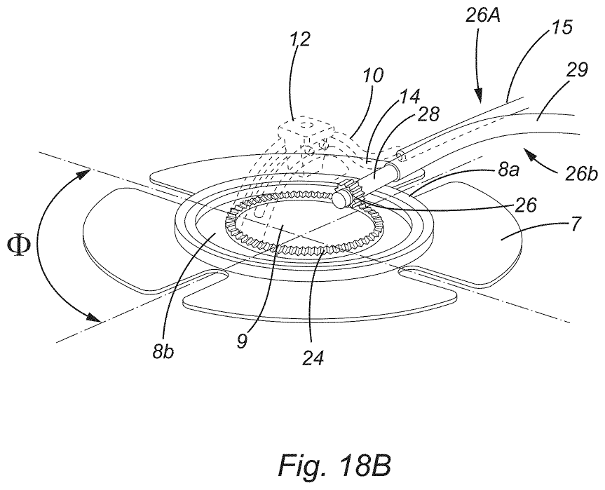

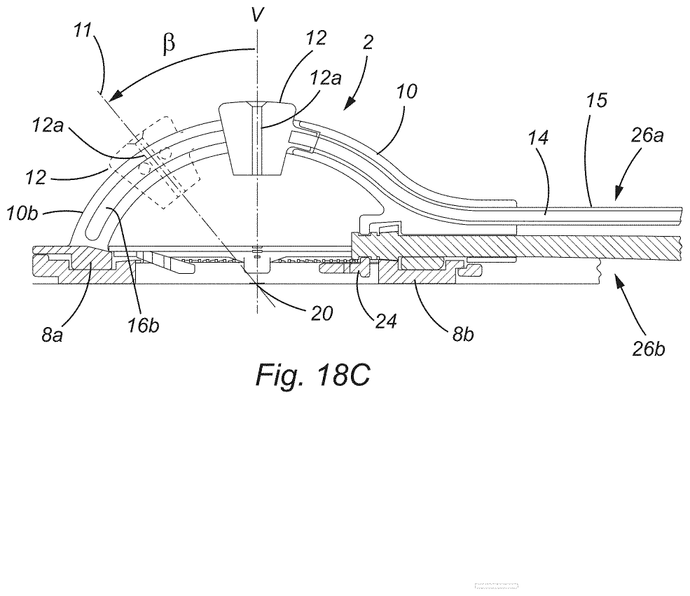

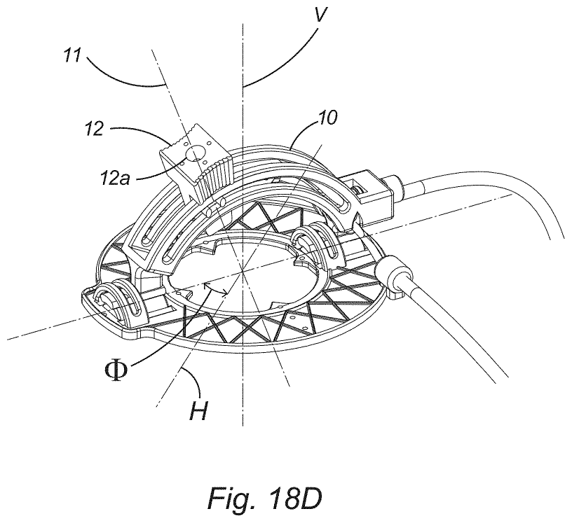

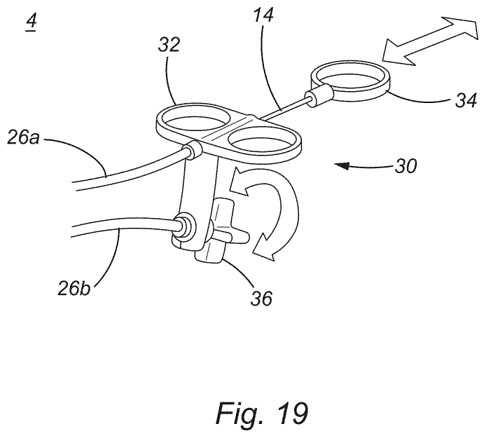

FIGS. 18A-19 show another embodiment of the disclosure. Similar features with the previous embodiments will be identified by the same element numbers. According to this embodiment, the assembly includes a base plate 8. Base plate 8 consists of an upper plate or ring 8a and lower plate or ring 8b. Lower plate 8b is fixed to the skin of a patient by adhesive patch 7. Upper plate 8a is rotatable with respect to the fixed lower plate 8b. According to one embodiment, contacting surfaces of plates 8a and 8b are provided with a coating or surface treatment that creates static friction so that the rotational position of the upper plate 8a remains fixed relative to the lower plate 8b, and hence, with respect to the patient's tissues, until force is applied to rotate upper plate 8a with respect to lower plate 8b.

Base plate 8 has a central opening 9. As with the previous embodiments, insertion point 20 is at the geometric center of upper plate 8a within opening 9.

Arch 10 is fixed with upper plate 8a. Arch 10 extends upward from plate 8a along a semicircular path and defines a plane perpendicular to the plane of plate 8a. Arch 10 has a constant radius centered on insertion point 20. According to the embodiment in FIG. 18A, arch 10 connects with upper plate 8a at both ends. Alternatively, arch 10 connects with plate 8a at only one end. Arch 10 supports a moveable guide body 12. According to this embodiment, instead of providing hinges between arch 10 and base 8, the arch is rigidly fixed with upper plate 8a and extends in a plane normal to the plane of plate 8a.

Guide body 12 is slidably positioned along arch 10. According to one embodiment, arch 10 includes segments 10a, 10b separated by a gap. Segments 10a, 10b each include a respective slots 16a, 16b. Guide body 12 is positioned in the gap between segments 10a, 10b.

Guide body 12 may be the same as shown in FIG. 7A-D. Posts 13 extend from the sides of body 12 and engage with slots 16a, 16b of segments 10a, 10b of arch 10. According to one embodiment, two or more posts 13 are provided on each side of body 12 that engage with slots 16a, 16b so that body 12 has a fixed radial orientation with respect to arch 10. This arrangement keeps bore 12a of guide body 12 aligned with the radius of arch 10. Engagement between body 12 and segments 10a, 10b allows body 12 to slide along the segments while keeping the guide body at a fixed radial orientation with respect to arch 10. According to one embodiment, contacting surfaces of body 12 and segments 10a, 10b are selected to provide a small amount of static friction so that body 12 will maintain its position along arch 10 until force is applied to reposition the body. According to another embodiment, arch 10 and guide body 12 may have the same features as those shown in FIGS. 10A-C, and 17A-17E.

As with the previous embodiments, bore 12a is sized and shaped to conform to the outer surface of a medical instrument and may be coated with a low friction coating, for example, PTFE as described with previous embodiments. Alternatively, bore 12 may be provided with an insert, such as the inserts shown in FIGS. 8A-9D.

As shown in FIG. 18C, insertion path 11 is defined by the longitudinal axis of bore 12a and intersects insertion point 20. Arch segments 10a, 10b follow a constant radius path centered on insertion point 20. Insertion path 11 is at angle β with respect to the guide assembly 2. As guide 12 is moved along arch 10 the orientation of insertion path 11 varies. Because bore 12a is aligned with the radius of arch 10 centered on insertion point 20, the insertion path 11 always intersects insertion point 20. As with the previous embodiments, motion of guide body 12 along arch 10 adjusts the angle β of bore 12a.

Cable 26a controls the angle β in a manner similar to the arrangement for adjusting angle β in the embodiments described with respect to FIG. 3D. According to one embodiment, cable 26a is a Bowden cable consisting of an inner shaft 14 surrounded by a sheath 15. The distal end of sheath 15 is fixed with arch 10 and upper plate 8a. The distal end of shaft 14 is connected with guide body 12. Shaft 14 can move in the proximal and distal directions within sheath 15. A lubricious coating or materials may be provided between shaft 14 and sheath 15 to provide ease of motion. Motion of shaft 14 is communicated to guide body 12 so that the guide body is moved along an arc defined by arch segments 10a, 10b as shaft 14 moves proximally and distally with respect to sheath 15 to adjust angle β.

FIG. 18B shows a view of guide assembly 2 showing the mechanism for adjusting a horizontal angle Φ using a rotary control cable 26b. Cable 26b cause arch 10 to rotate about the vertical axis V to vary horizontal angle Φ. Because the orientation of guide assembly 2 is fixed with respect to the patient's skin surface and is not necessarily in any particular geographic orientation, angle Φ may not be in the geographic horizontal plane. In addition, the value of angle Φ is relative to an arbitrary starting configuration of assembly 2.

Rotary cable 26b consists of an inner rotatable axle 28 surrounded by housing 29. Distal end of housing 29 is affixed with arch 10 and with upper plate 8a. At the distal end of inner axle 28 is pinion gear 26. Rack gear 24 is fixed with lower plate 8b and is extends at least part way around the circumference of central opening 9. Pinion gear 26 engages with rack gear 24. Rotation of axle 28 with respect to housing 29 causes pinion gear 26 to rotate with respect to upper plate 8a. Engagement of rotating pinion gear 26 with rack 24 causes plate 8a carrying arch 10 and needle guide 12 to rotate about the vertical axis V to adjust the horizontal angle Φ.

FIG. 18D shows the orientation of insertion path 11 with respect to the vertical axis V and an arbitrarily selected horizontal axis H. A projection of the insertion path 11 onto the horizontal plane defines an angle Φ with respect to axis H. In the configuration shown in FIGS. 18A and 18B, clockwise rotation of axle 28 and pinion gear 26 causes plate 8a, and hence arch 10, guide body 12, and bore 12a to move counterclockwise with respect to lower plate 8b decreasing horizontal angle Φ of insertion path 11 with respect to the horizontal axis H. Counterclockwise rotation of axle 28 causes the insertion path 11 to move clockwise, increasing horizontal angle Φ.

As shown in FIG. 18A, one or more adhesive patches 7 are connected with the lower ring plate 8b. As with previous embodiments, patches 7 connect the guide assembly 2 with the skin of a patient being treated. According to one embodiment, a single continuous patch 7 is provided that consists of lobes 59. The lobes of patch 7 allow the patch to conform to curved surfaces of a patient's body, for example, a patient's abdomen. According to another embodiment, patch 7 includes features described with respect to FIGS. 4A-6.

FIG. 19 shows a perspective view of remote operator 4 according to an embodiment of the disclosure to adjust the orientation of insertion path 11 of the guide assembly 2 shown in FIGS. 18A-18D. Cables 26a and 26b connect operator 4 with guide assembly 2. Operator body 30 includes finger grips 32. Shaft 14 of cable 26a extends though operator body 30 and terminates with a thumb grip 34. Sheath 15 of cable 26a is fixed to body 30. Grips 32, 34 are sized and shaped to allow a practitioner to hold body 30 and move shaft 14 in the proximal and distal directions with respect to sheath 15 as shown by the upper arrow in FIG. 19 using one hand.

As described above, when shaft 14 is moved in the distal direction, shaft 14 drives guide body 12 along arch 10 toward base 8 in the distal direction increasing angle β. When shaft 14 is moved in the proximal direction, guide body is moved proximally along arch 10 decreasing angle β. By pulling or pushing thumb grip 34 relative to finger grips 32, the practitioner adjusts angle β of the insertion path 11. According to one embodiment, graduation markings are provided on shaft 14 where it exits from operator body 30 to provide the practitioner with a numerical reading of the angle β of insertion path 11.

According to some embodiments, static friction between guide body 12 and arch 10 maintains the angle β of insertion path 11 until the practitioner applies force via grip 34 to reposition the guide body. According to other embodiments, a locking mechanism is provided on operator body 30, such as a compressive lock nut to releasably fix shaft 14 with respect to operator body 30 and sheath 15, so that once a desired position of guide body 12 along arch 10 is selected, guide body 12 can be fixed with respect to arch 10.

Axle 28 of rotary control cable 26b extends from the cable though operator body 30 and terminates at its proximal end with knob 36. Housing 29 of cable 26b is fixed with operator body 30. Rotation of knob 36, as shown by the lower arrow of FIG. 19, causes axle 28 to rotate with respect to axle housing 29. As shown in FIG. 18B, this rotation causes pinion gear 26 to rotate against rack gear 24 to drive upper plate 8a to rotate about the vertical axis V. Arch 10, which is supported by upper plate 8a is thus moved to adjust the horizontal angle Φ of the insertion path 11 defined by bore 12a.

The arrangement of arch 10 and rotatable upper plate 8a enables guide body 12 to move along two orthogonal planes. This allows a linear insertion path 11 defined by the bore 12a and passing through insertion point 20 to be selected by the practitioner by operating the operator body 30 through at least a portion of a hemisphere within the patient's tissue centered on the insertion point 20. Because this motion is communicated by cables 26a, 26b, the practitioner can adjust the insertion path while using an imaging device, such as a CT scanner from a safe location, for example, behind a radiation protective wall. As discussed with previous embodiments, the length of cables 26a and 26b is selected to conveniently allow the practitioner to operate the guide assembly from a safe distance. According to one embodiment, the length of cables 26a, 26b is between about 30 cm and 1000 cm. More preferably, the length of cables 26a, 26b is between about 100 cm and 800 cm. Most preferably, the length of cables 26a, 26b is about 140 cm.

In addition, because guide assembly 2 can be operated remotely, the space required to adjust insertion path 11 within an imaging device does not need to accommodate the practitioner's hands, potentially allowing a patient to be treated using an imaging device with a smaller bore. Also, cables 26a, 26b can be made from flexible materials so that unintentional motion by the practitioner is not communicated to guide assembly 2.

According to a further embodiment, a single cable communicates both rotational motion to a pinion gear 26 as described above with respect to rotary cable 26b and linear motion to guide body 12 via a sliding shaft 14, as described with respect to Bowden cable 26a. According to one embodiment, shaft 14 is arranged along the axis of rotary cable 26b.

A method of using needle guide 1 in conjunction with an imaging system 50 to facilitate insertion of a medical instrument into a patient is described according to one embodiment of the disclosure. A practitioner uses imaging systems 50 to provide a three-dimensional scan of the patient's tissues to determine a planned insertion trajectory for a medical instrument. The planned trajectory includes an identified insertion point where the instrument will enter the patient's body and a linear path from the insertion point to the targeted tissue. The planned trajectory may be stored as digital data as part of a planning scan. At the beginning of the procedure the planning scan is overlaid onto the new scans of the patient to confirm the incision point and the planned trajectory. According to some embodiments, instead of performing a planning scan to select a planned insertion path, the insertion path is determined once the guide assembly is in place on the patient's skin. This alternate method may reduce the time required for a procedure and may reduce the exposure of the patient to ionizing radiation.

Assembly 2 is fixed onto the patient with insertion point 20 centered on the incision point identified by the practitioner. Some imaging systems include laser alignment systems that project an alignment image on the patient's skin at the planned insertion point. According to some embodiments, center alignment features 44 include elements, such as holes 44b, 46c, are used in conjunction with the laser projection to align insertion point 20 of assembly 2 with the planned incision point determined by the practitioner. Because no ionizing radiation is required during this step, exposure for the patient and the practitioner is minimized.

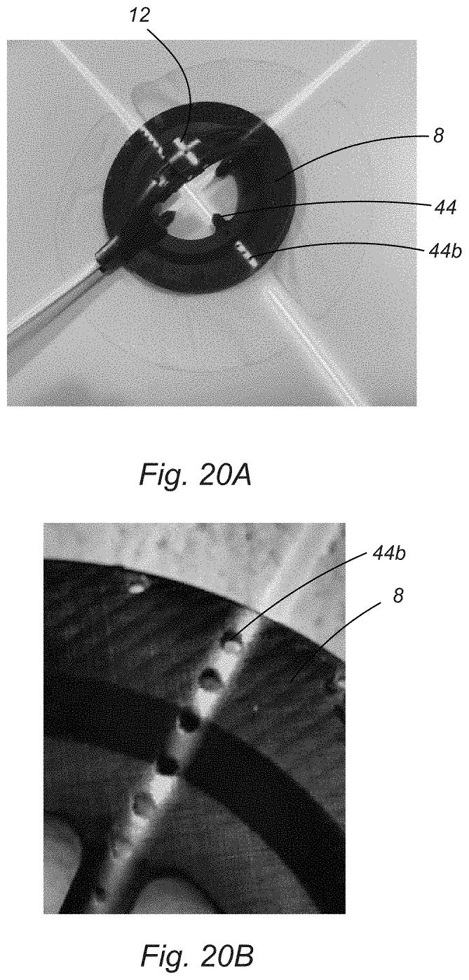

FIGS. 20A and 20B illustrate a guide assembly 2 according to embodiment of the disclosure positioned with the aid of a laser alignment system. In this embodiment, a series of holes 44b are provided on base 8 of the assembly. The holes are arranged co-linear with the center alignment features 44. The holes provide high contrast features when the base is illuminated by the laser. The practitioner adjusts the position of the assembly 2 until the holes line up with the projected laser scans. The practitioner then removes the protective covering from patch 7 and adheres assembly 2 to the patient.

Once assembly 2 is centered with insertion point 20 aligned with the incision point determined by the practitioner and adhered to the patient with patch 7, guide body 12, and arch 10 are adjusted to align insertion path 11 through bore 12a of guide body 12 with the planned trajectory. One or more repeat scans may be taken to confirm the device aligns with digital path. According to one embodiment, path alignment features 46a, 46b along bore 12a are used to visualize the insertion path relative to the targeted tissue. In addition, alignment features 46c-46g, illustrated in FIGS. 17A-17E, on the top surface of guide body 12 may be used to visualize the position of the laser alignment beams with guide body 12.

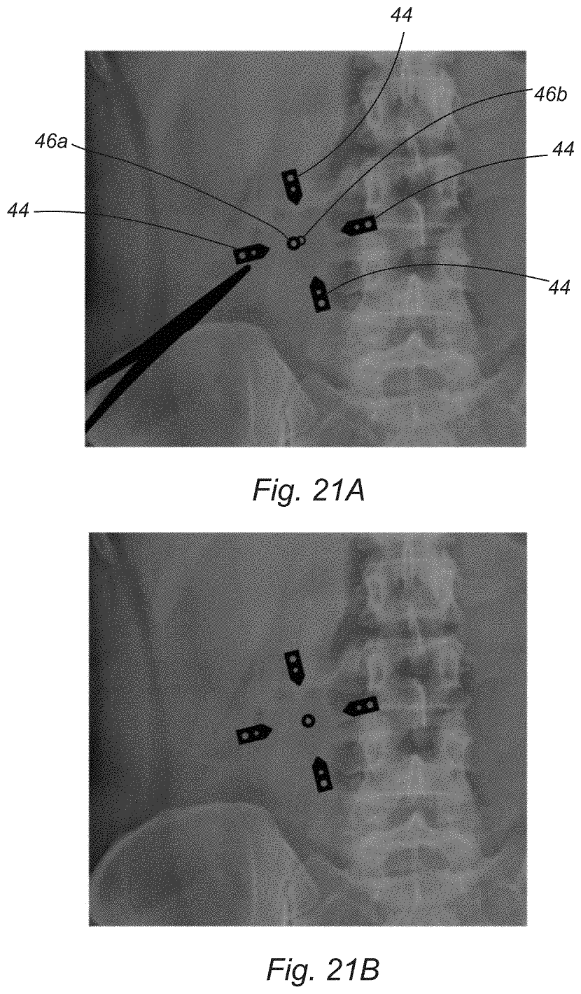

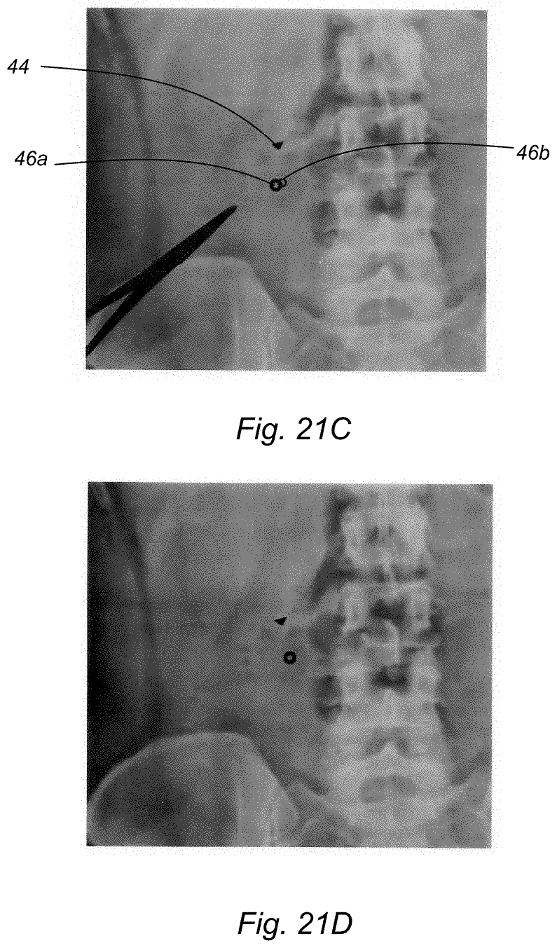

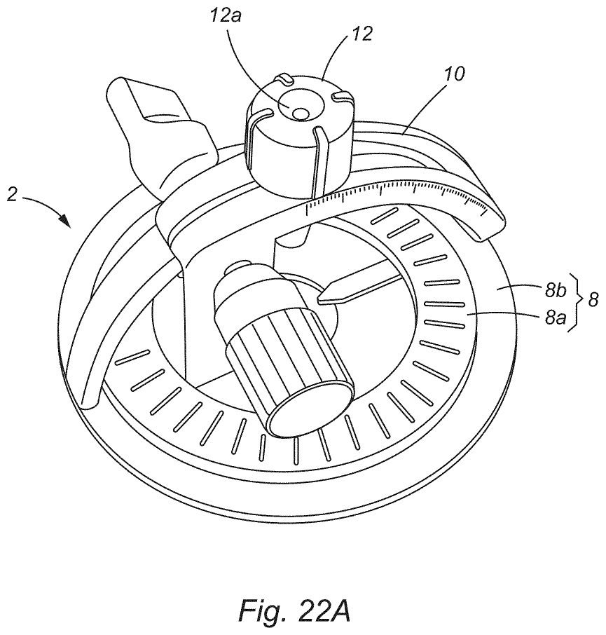

FIGS. 21A-21D illustrate how alignment features 44, 46a, 46b according to embodiments of the disclosure can be used in conjunction with a fluoroscopic imaging system. Most of the guide assembly 2 is radio-transparent so as not to obscure the view of the patient's tissue. Only the radio-opaque alignment features 44, 46a, 46b can be seen in these figures.

In FIG. 21A, assembly 2 has been affixed to a patient's skin. The view of the fluoroscope is arranged along the planned insertion trajectory with the insertion point centered in the imaging field. Four center alignment features 44 indicate the location of the insertion point. In the image in FIG. 21A, upper and lower path alignment features 46a, 46b arranged along bore 12a are not concentric, indicating the insertion path 11 is not aligned with the planned insertion trajectory.

FIGS. 21C and 21D show the positioning an alignment of assembly 2 where, instead of four center alignment features 44, only a single center alignment feature 44 is provided. By reducing the number of opaque features of assembly 2, this embodiment provides a clearer field of view for a practitioner to visualize potentially diseased tissue within the patient's body while adjusting insertion path 11.

While viewing the fluoroscopic image, the practitioner adjusts the insertion trajectory 11, for example, using remote operators 4a, 4b, until the upper path alignment feature 46a is concentric with the lower path alignment feature 46b so that insertion path 11 is aligned with the axis of the imaging system. Because the tissue being treated is visible in the image, the practitioner can ensure that the insertion path 11 intersects with the targeted tissue (e.g., a suspected tumor to be biopsied), as shown in FIGS. 21B and 21D. Because the guide assembly 2 can be adjusted some distance from the imaging system, for example as shown in FIG. 1, the practitioner can align insertion path 11 while remaining physically distant from the imaging system, thus minimizing the practitioner's exposure to ionizing x-ray radiation.

Once the insertion path is confirmed as correct, the practitioner locks assembly 2, for example, by releasing downward pressure on actuators 140 of remote operators 4a, 4b as illustrated in FIGS. 14A-14C so that actuators 140 engage with toothed rails 46 to prevent unintentional movement or deviation from the selected trajectory. Once assembly 2 is locked, the patient may be removed from the imaging device to perform the procedure. The practitioner may create a small incision at insertion point 20 to facilitate insertion of an instrument into the patient. Because arch 11 and base 8 are spaced away from the skin of the patient, the practitioner has sufficient clearance to reach in and make the incision. According to one embodiment, bladed instrument, such as a lancet or scalpel disposed on a shaft sized to pass through bore 12a is provided. This instrument is advanced through guide body 12 until it contacts incision point 20 to create the incision. The bladed instrument is then withdrawn from bore 12a.

The practitioner can then insert the needle, introducer, or other medical instrument to be used to perform the procedure through bore 12a along insertion path 11. According to some embodiments, repeat scans can be performed to confirm the needle position at stages of insertion. Because the device fixes the insertion path 11, a smaller number of repeat scans may be required, thus reducing the exposure of the patient and medical personnel to ionizing radiation.

According to one embodiment, once the procedure is complete and the medical instrument is withdrawn from the patient, arch 10 and guide body 12 are used to position and stabilize a bandage, sponge, or other material against the wound. Patch 7 remains fixed to the patient's skin following the procedure. According to this embodiment, a compressible, absorbent material, such as a gel foam sponge sized with an uncompressed size somewhat larger than the space beneath the arch is squeezed beneath the arch so that it is pressed against the wound. This embodiment may allow bleeding of the wound to be staunched without requiring a medical professional to apply pressure. This embodiment may also allow the patient to be moved from the surgical suite without needing to wait until bleeding from the wound has stopped.

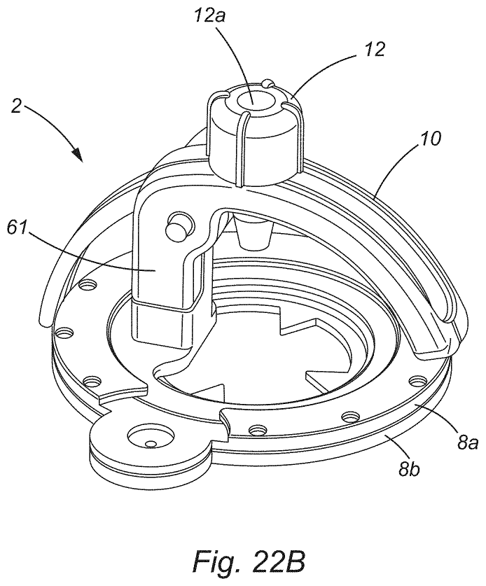

FIGS. 22AA, 22B, 23, and 24 show a guide assembly 2 according to a further embodiment of the disclosure. As shown in FIGS. 22A and 22B, guide assembly 2 includes a base 8 comprised of a lower plate 8b that is positioned on the patient's skin and fixed at a selected location relative to a planned insertion point and an upper plate 8a. Upper plate 8a rotates with respect to lower plate 8b.

Arch 10 is connected with rotatable upper plate 8a by post 61. Guide body 12 is slidably mounted on arch 10. As with the previous embodiments, arch 10 extends, at least partially, along a semicircular path with the insertion point 20 at the center of curvature of arch 10. Guide body 12 includes a bore 12a. As with previous embodiments, bore 12a is sized to allow insertion of a needle or other medical instrument along a selected insertion path 11 through insertion point 20 and into the patient's tissues.

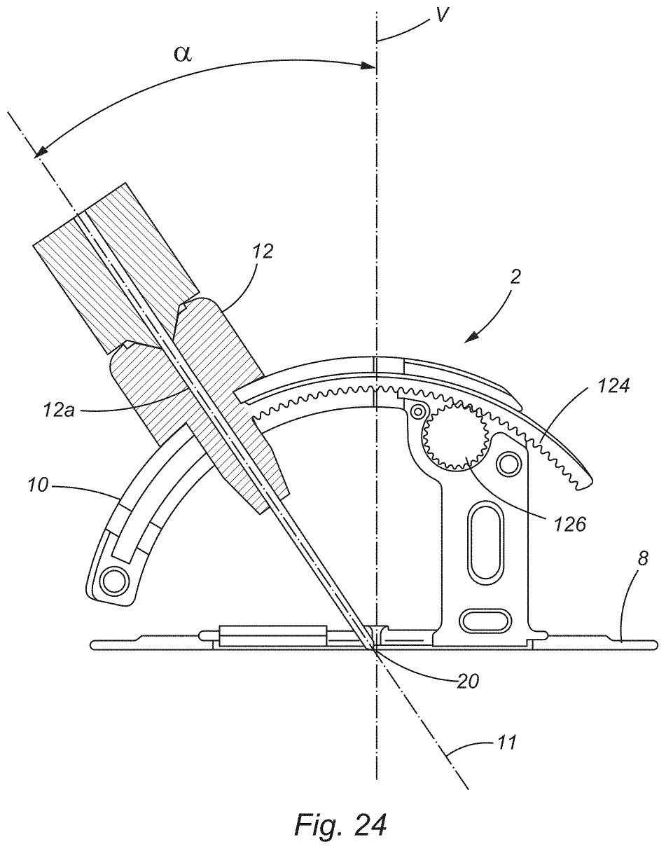

Control knob 60 allows adjustment of the insertion path 11 by moving guide body 12 along arch 10. FIG. 24 shows a cross section of guide assembly 2 at a plane through the center of arch 10. Knob 60 is connected with pinion gear 126. Pinion gear 126 engages with rack gear 124. Rack gear 124 extends along an internal channel of arch 10 and is connected with guide body 12. Rotation of knob 60 turns pinion gear 126 and drives guide body 12 along arch 10 to adjust the angle β of insertion path 11 relative to the vertical axis V. According to one embodiment, arch 10 includes markings indicating the angle β.

Contacting surfaces between guide body 12 and arch 10 may be selected to provide static friction that holds the guide body in a fixed position with respect to the arch until force is applied via pinion gear 126 to move the guide body. According to another embodiment, guide body 12 includes a locking mechanism operable to fix it in position along arch 10. According to one embodiment, an outer portion of guide body 12 has an internal thread that engages with an inner portion of the guide body. Rotation of the outer portion tightens the outer potion against the arch, fixing the guide body 12 in position along the arch 10.

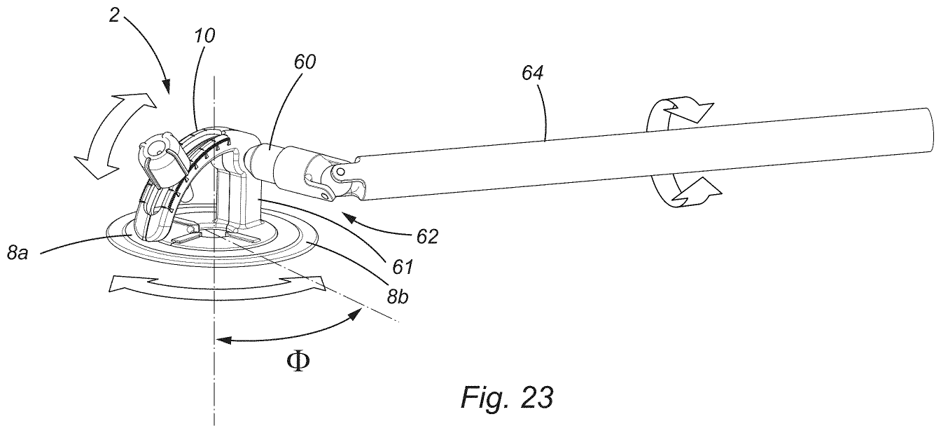

As shown in FIG. 23, guide assembly 2 can be coupled with a control arm 64 that allows the insertion path 11 to be adjusted remotely. A universal joint 62 is provided between control arm 64 and control knob 60. Rotation of arm 64 is communicated via joint 62 to rotate knob 60. Proximally and distally directed force applied to arm 64 causes plate 8a to rotate with respect to plate 8b. Arch 10 connected with the rotatable upper plate 8a likewise rotates to adjust the horizontal angle Φ of insertion path 11. According to one embodiment, universal joint 62 removably couples with knob 60. According to other embodiments, universal joint 62 permanently fixes arm 64 with assembly 2. The embodiment shown in FIG. 23 has a single universal point 62. According to other embodiments, two or more universal joints may be provided between arm 64 and knob 60 to provide addition flexibility.

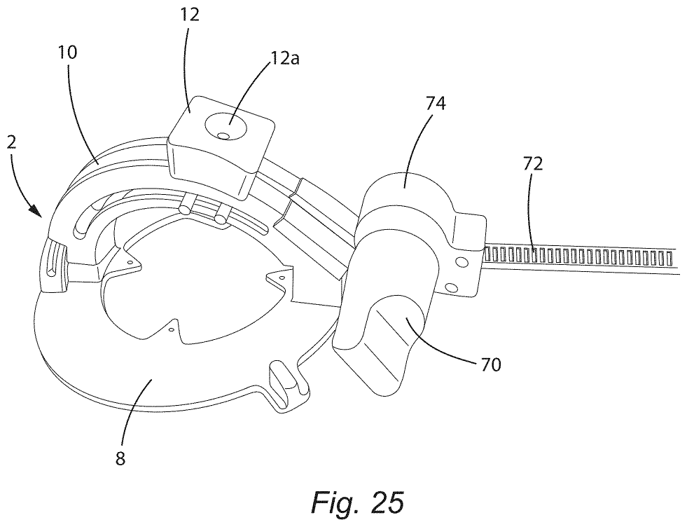

FIG. 25 shows another embodiment of guide assembly 2. Guide body 12 is slidably engaged with arch 10. Bore 12a defines an insertion path 11, as described in the previous embodiments. Base plate 8 supports arch 10 via sliding hinges, such as those described with respect to FIGS. 3A-3D, to allow arch 10 to swing about a hinge axis. Base plate 8 is fixed to the patient's skin using an adhesive patch, such as patch 7 described above.

In this embodiment, a flexible rack 72 extends through a channel in arch 10. One end of rack 72 is connected with guide body 12. The other end of rack 72 extends outward from assembly 2. Gear assembly 74 is connected with arch 10. Teeth of a gear (not shown) within the gear assembly 74 engage with teeth of the rack 72. Gear assembly 74 is connected with knob 70 that extends from assembly 2. Rotation of the knob causes the gear within gear assembly 74 to rotate and to cause flexible rack 72 to move toward and away from arch 10, displacing guide body 12 along arch 10. According to some embodiments, knob 70 is connected with an arm, such as arm 64 as described with respect to FIG. 23, to allow remote adjustment of the insertion path 11 by moving guide body 12 along arch 10.

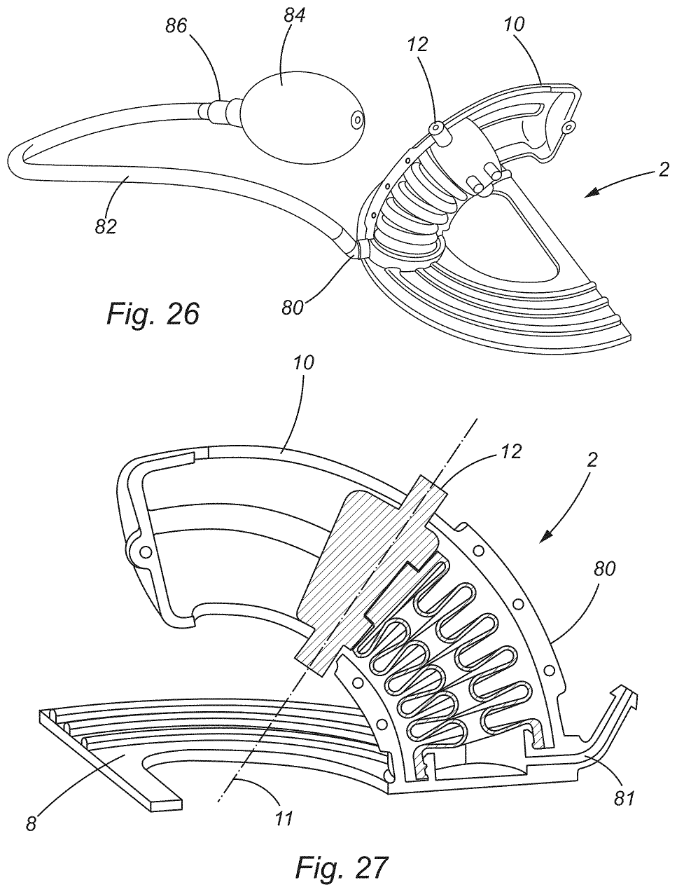

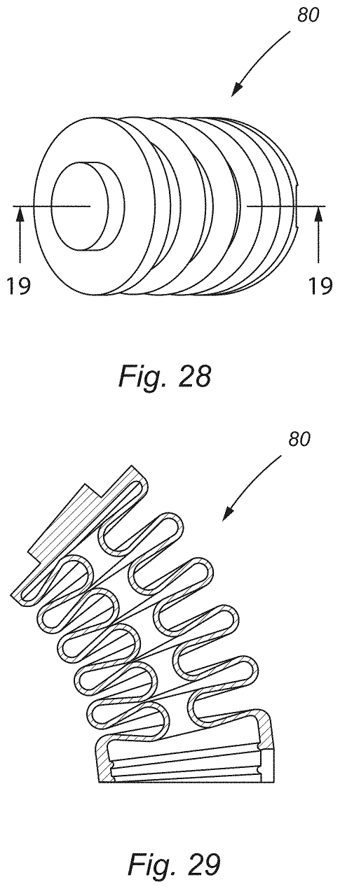

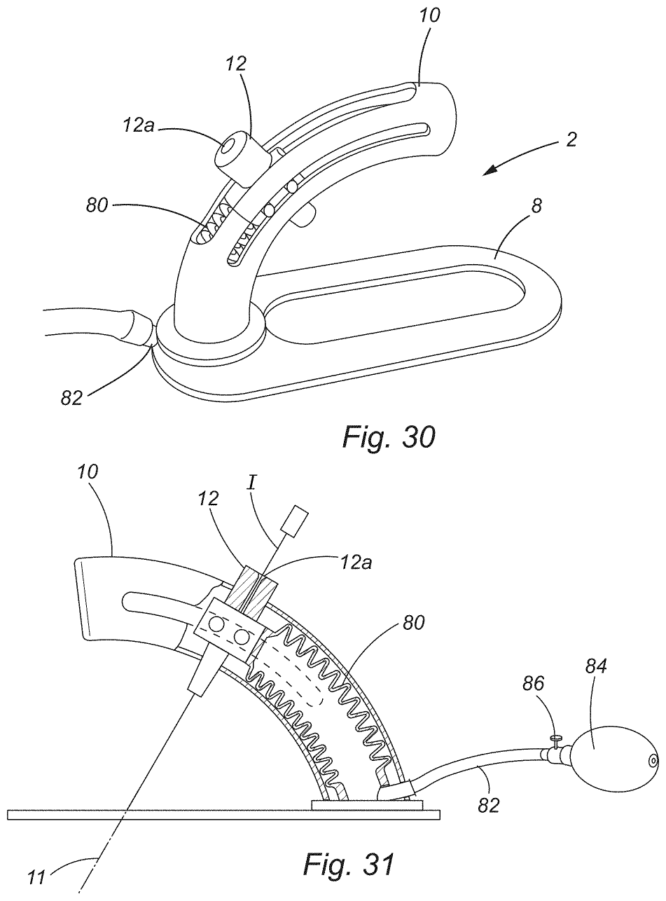

FIGS. 26-31 show a guide assembly 2 according to a further embodiment of the disclosure. Base plate 8 is fixable to the patient's skin, for example, using patch 7 as disclosed in previous embodiments. Plate 8 supports arch 10. Guide body 12 is slidably mounted to arch 10 and moves along arch 10 to adjust insertion path 11. Arch 10 includes a hollow interior space housing a bellows 80. The lower end of bellows 80 is sealed to a manifold 81 so that fluid pressure applied to the manifold causes bellows 80 to inflate and expand. FIGS. 28 and 29 show detailed view and a cross section view, respectively, of bellows 80. The upper end of bellows 80 is fixed with guide body 12.

As shown in FIG. 26, manifold 81 is connected with tube 82. At a proximal end of tube 82 is squeeze bulb 84 and valve 86. Opening valve 86 causes gas within tube 82 and bellows 80 to vent, bringing the pressure in bellows 80 to atmospheric pressure. When valve 86 is closed, pressure applied to bulb 84 displaces air along hose 82 applying pressure to expand bellows 80. As bellows 80 expands, guide body 12 is moved along arch 10. Expansion of bellows 80 adjusts angle β of insertion path 11. Valve 86 may be provided with a further closure mechanism to isolate bulb 84 from tube 82. According to one embodiment, when the desired insertion path 11 has been selected, the further closure mechanism of valve 86 is operated, fixing the pressure within bellows 80 and fixing the angle β of insertion path 11.

Bellows 80 is molded so that, when no internal pressure is applied, the bellows resiliently assumes a contracted configuration, as shown in FIGS. 28 and 29. When valve 86 is opened, air is allowed to escape bellows 80 via tube 82 causing bellows 80 to contract and moving guide body 12 downward along arch 10. By alternately applying and releasing pressure within bellows, the orientation of guide body 12, and hence insertion path 11, can be adjusted. As shown in FIG. 29, bellows may be molded to have a curved shape to follow the curvature of arch 10.

Tube 82 is flexible so that motion of bulb 84 is not communicated to assembly 2, reducing the chance that the assembly will be disturbed by unintentional motion by the practitioner. The length of tube 82 can be selected to allow the practitioner to operate the device at a distance, for example, to avoid exposure to ionizing radiation as discussed above for previous embodiments.

Instead of air being displaced from bulb 84 to inflate bellows 80, another gas could be used. Also, instead of using pneumatic pressure, a hydraulic fluid could be provided to expand bellows 80.

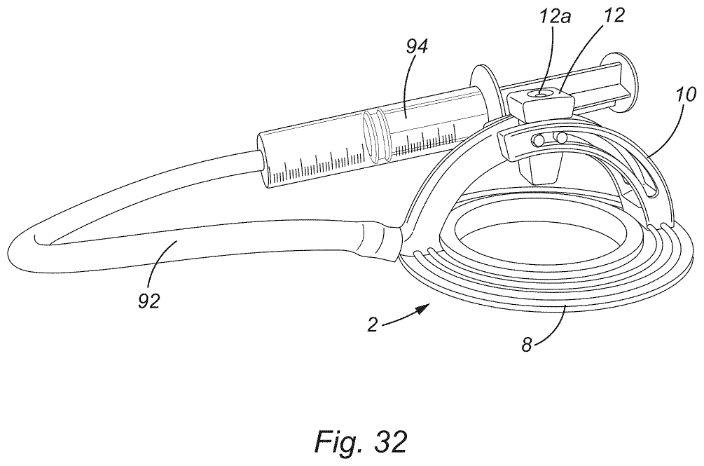

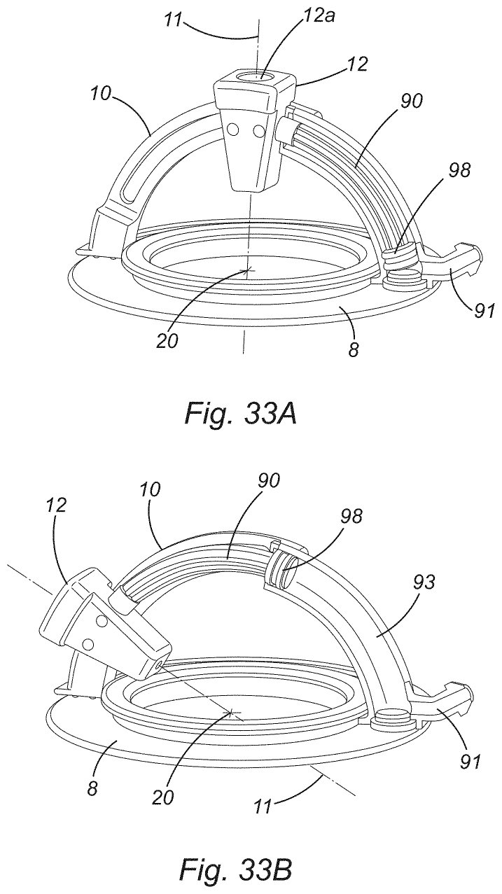

FIGS. 32, 33A and 33B show a guide assembly 2 according to a further embodiment of the disclosure. Base 8 supports arch 10. Base 8 may be formed from a lower plate 8b that is fixed to a patient's skin and an upper, rotatable plate 8a that can be rotated by a practitioner to adjust a horizontal angle of insertion path 11 as in the embodiment of FIGS. 18A-18D. Alternatively, arch 10 may be connected with base 8 by sliding hinges, as shown for example, in the embodiments described with respect to FIGS. 3A-3D.

Guide body 12 is slidably mounted to arch 10. As with previous embodiments, bore 12a defines insertion path 11. Arch 10 follows a semicircular arc with a radius of curvature centered on an insertion point so that the insertion path 11 intersects the insertion point throughout the motion of guide 12 along arch 10. According to one embodiment arch 10 and guide body 12 are configures as shown in FIGS. 10A-10C.

FIGS. 33A and 33B show cross sections of arch 10. A cavity 93 is formed on the interior of arch 10. Piston 90 fits within cavity 93 and slides along the length of the cavity. A distal end of piston 90 is connected with guide 12. A proximal end of piston 90 remains within cavity 93. One or more fluid-tight seals 98 are provided between the inner wall of cavity 93 and the outer surface of piston 90. Seals 98 slide along the wall of cavity 93.

At the proximal end of cavity 93 is a connector 91. As shown in FIG. 32, distal end of hose 92 is connected with connector 91. A syringe 94 is connected with the proximal end of hose 92. A hydraulic fluid fills the space within syringe 94, hose 92, and cavity 93.

As shown in FIG. 33B, when syringe 94 is pressed, fluid is driven through hose 92 into cavity 93, displacing piston 90 and driving guide body 12 in the distal direction along arch 10, increasing the angle β of insertion path 11. When syringe 94 is pulled, fluid is withdrawn from cavity 93, pulling piston 90 in the proximal direction and pulling guide 12 upward along arch 10 and decreasing the angle β.

Graduations may be provided on syringe 94 corresponding to the vertical angle of insertion path 11. As with previous embodiments, hose 92 is flexible so that unintentional movement by the practitioner operating the syringe 94 is not communicated to assembly 2. The length of hose 92 may be selected to allow the practitioner to operate the assembly at a safe distance from ionizing radiation, for example, from a fluoroscope used to visualize the insertion path 11.

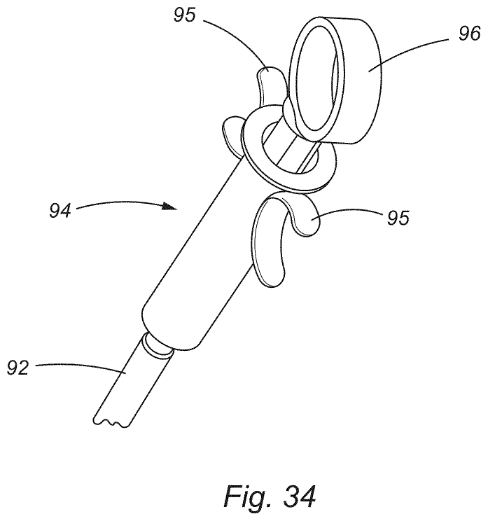

FIG. 34 shows a syringe 94 according to an embodiment of the disclosure. As in the previous embodiment, syringe 94 is connected with hose 92 to move fluid into and out of cavity 93. According to this embodiment, syringe includes one or more finger grips 95 and a thumb grip 96. Grips 95, 96 make it easier for the practitioner to make fine adjustments to the amount of fluid driven into cavity 93, and hence to make fine adjustments to the orientation of insertion path 11.

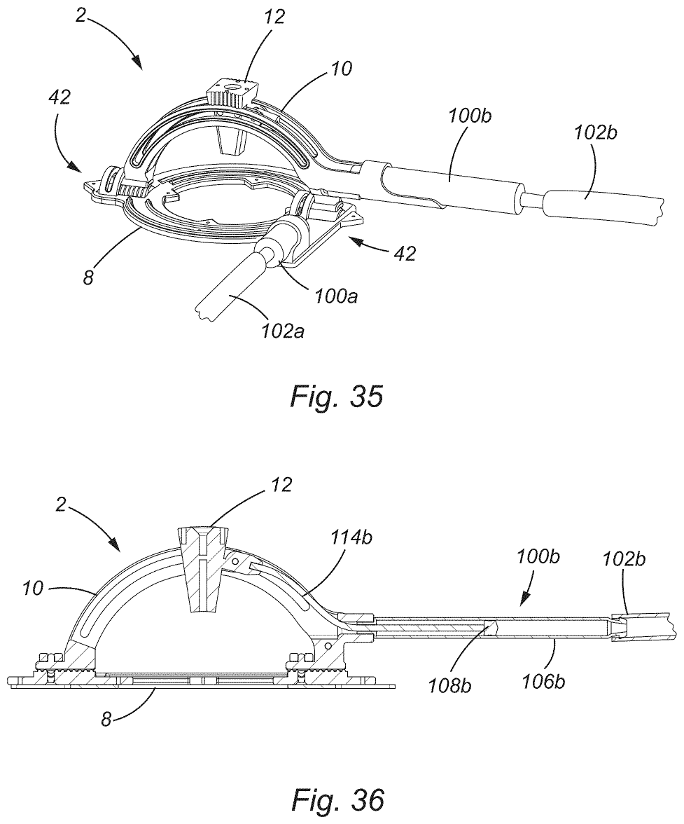

FIGS. 35, 36, and 37 show guide assembly 2 according to another embodiment of the disclosure. Base 8 is fixed to the skin of the patient using, for example, adhesive patch 7 as described in previous embodiments. Arch 10 is connected with base 8 by hinges 42. Hinges 42 may be sliding hinges, as described above with respect to FIGS. 3A-3D. Guide body 12 is slidingly mounted to arch 10 so that bore 12a defines an insertion path 11 as with embodiments described above. Arch 10 and guide body 12 may be configured as shown in FIGS. 10A-10C.

In this embodiment, actuator rods 114a and 114b are connected with arch 10 and with guide body 12, respectively. Motion of rods 114a, 114b cause the arch 10 and needle guide 12 to move with respect to base 8 to adjust insertion path 11. Rods 114a, 114b are driven by hydraulic actuators 100a, 100b.

Actuator 100b drives guide body 12 along arch 10 to adjust angle β of insertion path 11. FIG. 36 is a cross section of assembly 2 showing the mechanism of actuator 100b. Actuator 100b is connected at its proximal end with hose 102b. Hydraulic chamber 106b is in fluid communication with hose 102b. Plunger 108b is fitted within chamber 106b and include seals that allow the plunger 108b to slide within chamber 106b in response to fluid moved into or out of the chamber. Actuator rod 114b is connected with the distal side of plunger 108b. Distal end of actuator 100b is fixed to arch 10. A syringe, such as the syringe shown in FIG. 34, is connected with the proximal end of hose 102b. The space within syringe and within hose 102b and hydraulic cavity 106b is filled with a hydraulic fluid. When the syringe is pushed, hydraulic fluid is displaced through hose 102b, into chamber 106b, driving plunger 108b and the attached rod 114b in the distal direction to move guide body 12 along arch 10. When the syringe is pulled, fluid is withdrawn through hose 102b, pulling plunger 108b and the attached rod 114b in the proximal direction.

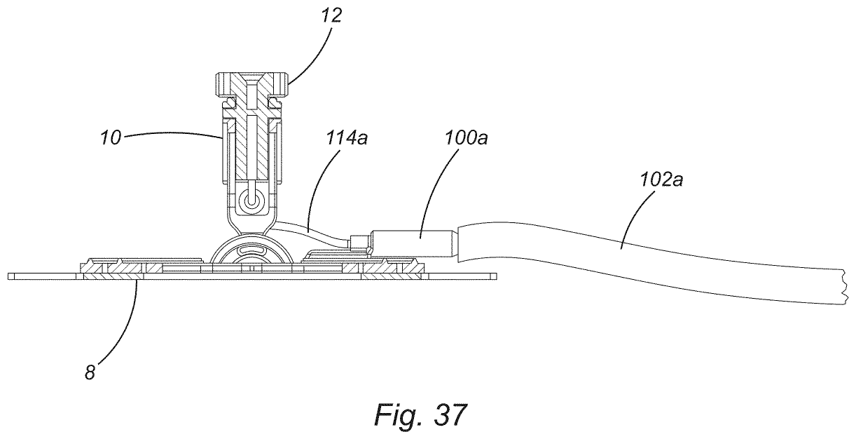

As shown in FIG. 37, the distal end of actuator 100a is connected with base 8 and rod 114a extends from the actuator to connect with arch 10. Hose 102a connects with actuator 100a. As with the embodiment described with respect to FIG. 36, a chamber, and plunger arrangement are provided with actuator 100a. A syringe, such as the syringe shown in FIG. 34 is connected with the proximal end of hose 102a. The space within the syringe, hose 102a, and the chamber of actuator 100a are filled with a hydraulic fluid. Pushing and pulling the syringe drives fluid into and out from the chamber of actuator 100a, driving rod 114a to move arch 10 about the axis of hinges 42 to adjust angle α.

Hoses 102a, 102b can be made as long as necessary to allow assembly 2 to be adjusted from a safe distance to reduce the exposure of the practitioner to ionizing radiation. Because hoses 102a, 102b are flexible, assembly 2 is isolated from unintended motion by the practitioner.

According to another embodiment, instead of actuators 100a, 100b driven by hydraulic or pneumatic pressure as shown in FIGS. 35-37, electrical motors are provided. The motors are connected with rods 114a, 114b. The motors displace rods 114a, 114b to change the orientation of arch 10 and guide body 12 to adjust insertion path 11. According to this embodiment, instead of hoses 102a, 102b, electrical wires are provided to deliver power to the motors. An electrical controller is connected with the wires. A practitioner operates the electrical controller to drive the motors to change the orientation of insertion path 11. According to a further embodiment, the motors include a self-contained power source, such as a rechargeable battery, and a communication interface, such as a Bluetooth™ transceiver. The practitioner sends signals to the motors from a communication device also equipped with a Bluetooth™ transceiver, such as a computer tablet, to energize the motors to change the orientation of arch 10 and guide body 12.

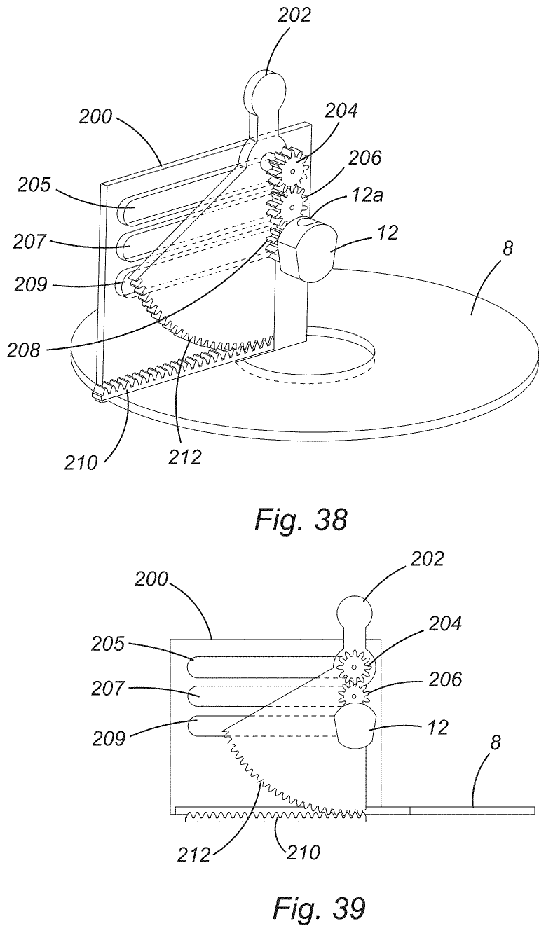

FIGS. 38, 39, and 40 show another embodiment of the disclosure. Needle guide 12 includes bore 12a, as discussed in previous embodiments. Base plate 8 may be fixed to the skin of the patient. Base plate 8 may include a fixed portion and a rotatable portion as described with respect to previous embodiments to allow the horizontal angle of the path of insertion to be adjusted.

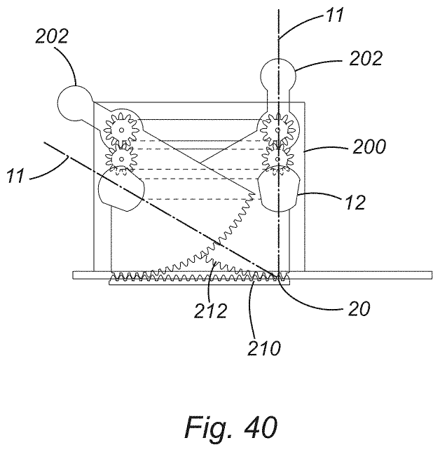

Cam support 200 is fixed to base plate 8. Lever arm 202 is slidably connected with support 200 so that the lever arm 202 can move along the face of support 200. FIG. 40 shows lever 202 moved from a first orientation where path of insertion 11 is vertical to a second orientation where the path of insertion is oblique to the vertical axis.

First gear 204 is fixed to lever arm 202. A drive gear 212 is provided along the lower edge of lever arm 202. Rack gear 210 is fixed to base plate 8. Drive gear 212 engages with rack gear 210 so that, when leaver 202 is moved from right to left along the face of support 200 (as shown in the orientation of FIG. 40), lever arm 202 rotates in the counterclockwise direction. First gear 204, fixed to arm 202, likewise rotates in the counterclockwise direction.

Second gear 206 is connected with arm 202 but is free to rotate. Second gear 206 is engaged with first gear 204. Counterclockwise rotation of first gear 204 causes second gear 206 to rotate clockwise. Third gear 208 is also connected with arm 202, is engaged with second gear, and is free to rotate. Needle guide 12 is fixed to third gear 208. When second gear 206 rotates clockwise, third gear 208 rotates counterclockwise, causing needle guide to likewise rotate counterclockwise and to change the angle of the path of insertion 11.

As lever arm 202 is moved from right to left along the face of support 200, needle guide 12 likewise moves from right to left. The ratio of gears 204, 206, 208, 210, and 212 are selected so that, as needle guide 12 translates along the face of support 200, the angle of the path of insertion 11 (defined by bore 12a of needle guide 12) always intersects insertion point 20. By moving lever arm 202 with respect to support 200, the vertical angle of the path of insertion 11 is adjusted, while maintaining a fixed point of insertion 20.

According to some embodiment, instead of, or in addition to manual actuators, one or more electric motors are provided to drive mechanisms on guide assembly 2 to change the orientation of insertion path 11. Such motors may be controlled by wires connected with a controller and power source to energize the motors to move guide body 12 along arch 10 and/or to change the orientation of arch 10 or base 8 to adjust insertion path 11. Alternatively, such motors are provided with a power source, such as a battery, and with a radiofrequency communication device, such as a Bluetooth™ transceiver. Control signals generated by a remote computing device, such as a computer tablet operated by a practitioner, are received by the transceiver and used to control the motors to adjust the trajectory of insertion path 11.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

1. A medical device introducer guide comprising:
a guide assembly comprising:
a base adapted to be affixed to an organism relative to an insertion point;
an arch connected with the base, the arch having a semicircular curvature, the curvature having a radius of curvature centered on the insertion point, wherein the insertion point is co-planar with an outer surface of the organism; and
a guide body slidably disposed on the arch, the guide body including a bore, wherein an axis of the bore defines an insertion path, wherein the insertion path has a trajectory, and wherein the insertion path intersects the insertion point;
a remote operator; and
a linkage connected with the remote operator and the guide assembly, wherein a motion of the remote operator is communicated by the linkage to one or more of the arch and the guide body to vary the trajectory of the insertion path, where the linkage comprises a first cable, wherein the first cable comprises a first shaft and a first sheath surrounding the first shaft, wherein a distal end of the first sheath is fixed to the arch, wherein a distal end of the first shaft is fixed to the guide body, and wherein the motion is communicated by movement of the first shaft relative to the first sheath to move the guide body along the arch to vary the trajectory of the insertion path through a first angle.

2. The introducer guide of claim 1, further comprising one or more hinges connecting the arch with the base, wherein the one or more hinges allow the arch to rotate about an axis of rotation parallel with the base, and wherein the axis of rotation intersects the insertion point.

3. The introducer guide of claim 2, wherein the one or more hinges comprise two sliding hinges, the sliding hinges each comprising:
a semicircular support surface fixed to the base and having a hinge radius of curvature, wherein the hinge radius of curvature is centered on the axis of rotation; and
a slider having a sliding surface in sliding contact with the support surface, wherein the arch is fixed with the slider and extends from the slider in a direction radially away from the support surface, and wherein rotation of the arch about the axis of rotation slides the slider along the support surface.

4. The introducer guide of claim 3, wherein a curvature of the sliding surface conforms with the curvature of the support surface.

5. The introducer guide of claim 4, further comprising a retainer fixed with the base, wherein the retainer has a semicircular inner surface that is concentric with the support surface, wherein an upper surface of the slider is in sliding contact with the retainer, and wherein the retainer holds the slider against the support surface.

6. (canceled)

7. A medical device introducer guide comprising:
a guide assembly comprising:
a base adapted to be affixed to an organism relative to an insertion point;
an arch connected with the base, the arch having a semicircular curvature, the curvature having a radius of curvature centered on the insertion point, wherein the insertion point is co-planar with an outer surface of the organism; and
a guide body slidably disposed on the arch, the guide body including a bore, wherein an axis of the bore defines an insertion path, wherein the insertion path has a trajectory, and wherein the insertion path intersects the insertion point;
a remote operator; and
a linkage connected with the remote operator and the guide assembly, wherein a motion of the remote operator is communicated by the linkage to one or more of the arch and the guide body to vary the trajectory of the insertion path, wherein the linkage comprises a second cable, wherein the second cable comprises a second shaft and a second sheath surrounding the second shaft, wherein a distal end of the second sheath is fixed to the base, wherein a distal end of the second shaft is fixed to the arch, and wherein the motion is communicated by movement of the second shaft relative to the second sheath to move the arch relative to the base and to vary the trajectory of the insertion path through a second angle.

8. The introducer guide of claim 1, wherein the remote operator comprises an operator having a housing and a sliding actuator positioned in the housing and adapted to slide in a distal and a proximal direction relative to the housing to generate the motion communicated by the linkage to vary the trajectory of the insertion path.

9. The introducer guide of claim 8, wherein the remote operator further comprises a toothed rail fixed to the first housing, wherein the first sliding actuator comprises a ridged surface shaped to engage the toothed rail and a spring between the housing and the first sliding actuator, wherein resiliency of the spring holds the ridged surface of the actuator in engagement with the toothed rail to fix a position of the first sliding actuator relative to the housing, and wherein pressure applied to the first sliding actuator disengages the ridged surface from the toothed rail to allow the first sliding actuator to move in the proximal and distal directions.

10. The introducer guide of claim 1, wherein the first cable has a length between about 30 cm and 250 cm.

11. The introducer guide of claim 1, wherein the guide body further comprises a plurality of membranes arranged along the bore, wherein the membranes include openings arranged collinearly and aligned with the insertion path.

12. The introducer guide of claim 11, wherein one or more of the membranes comprise a plurality of resilient tines extending into the bore, wherein, when the tines are flexed by an instrument inserted along the bore, the tines are adapted to apply resilient force on the instrument toward the insertion path.

13. The introducer guide of claim 1, wherein the guide assembly comprises a material with a first radio-opacity and wherein the base further comprises one or more center alignment indicators shaped to indicate a direction relative to the insertion point, wherein the center alignment indicators have a radio-opacity greater than the first radio-opacity, and wherein, when viewed under x-ray radiation, the center alignment indicators show the position of the insertion point.

14. The introducer guide of claim 1, wherein the guide body comprises a plurality of path alignment indicators arranged co-linearly with the bore, wherein the path alignment indicators have a radio-opacity different from the first radio-opacity, and wherein, when viewed under x-ray radiation, the path alignment indicators show the trajectory of the insertion path.

15. The introducer guide of claim 1, wherein one or more of the base and the guide body comprise optical alignment features arranged to reflect light emitted by a laser alignment system of an imaging system and to provide a visual indication of the position of the base or the guide body with respect to the imaging system.

16. The introducer guide of claim 15, wherein the optical alignment features comprise one or more holes, extensions, or grooves on the guide body.

17. A medical device introducer guide comprising:
a guide assembly comprising:
a base adapted to be affixed to an organism relative to an insertion point;
an arch connected with the base, the arch having a semicircular curvature, the curvature having a radius of curvature centered on the insertion point, wherein the insertion point is co-planar with an outer surface of the organism; and
a guide body slidably disposed on the arch, the guide body including a bore, wherein an axis of the bore defines an insertion path, wherein the insertion path has a trajectory, and wherein the insertion path intersects the insertion point;
a remote operator; and
a linkage connected with the remote operator and the guide assembly, wherein a motion of the remote operator is communicated by the linkage to one or more of the arch and the guide body to vary the trajectory of the insertion path,
wherein the base comprises a lower plate and an upper plate, wherein a bottom surface of the lower plate adapted to be fix to the organism, wherein an upper surface of the lower plate comprises a rack gear disposed along at least part of a circular path centered on the insertion point, wherein the upper plate is rotatably connected with the lower plate, wherein the arch is fix to the upper plate and extends upward in a plane normal to the upper plate, wherein a pinion gear is rotatably mounted to the upper plate, wherein the pinion gear engages the rack gear, and wherein rotation of the pinion gear causes the upper plate and the arch to rotate relative to the lower plate.

18. The introducer of claim 17, wherein the linkage comprises a rotary cable, wherein a distal end of the rotary cable is connected with the pinion gear, wherein the remote operator comprises a knob connected with a proximal end of the rotary cable, and wherein rotation of the knob causes the upper plate and arch to rotate relative to the lower plate.

19. The introducer guide of claim 1, wherein the linkage further comprises one or more of a Bowden cable, a rotary control cable, a hydraulic cylinder, and a pneumatic cylinder.

20. The introducer guide of claim 1, wherein the linkage comprises a fluid-driven actuator, wherein, when fluid is moved into or out from the actuator, the actuator exerts force on the guide body to move the guide body to the selected location.
